SEQUENCE LISTING (12 sequences)

SEQ ID NO: 1 - DNA, 49 bp, synthetic construct
  source 1..49: mol_type = other DNA
aatgatacggcgaccaccgagatctacactctttccctacacgacgctc

SEQ ID NO: 2 - DNA, 18 bp, synthetic construct
  source 1..18: mol_type = other DNA
agggcacagccacatcct

SEQ ID NO: 3 - DNA, 17 bp, synthetic construct
  source 1..17: mol_type = other DNA
ttggtgttgctgggctt

SEQ ID NO: 4 - DNA, 18 bp, synthetic construct
  source 1..18: mol_type = other DNA
tgacgtccttggaagcca

SEQ ID NO: 5 - DNA, 18 bp, synthetic construct
  source 1..18: mol_type = other DNA
tgtgggacttccactggt

SEQ ID NO: 6 - DNA, 18 bp, synthetic construct
  source 1..18: mol_type = other DNA
tgacttcgcaggcataga

SEQ ID NO: 7 - DNA, 25 bp, synthetic construct
  source 1..25: mol_type = other DNA
aatgatacggcgaccaccgagatct

SEQ ID NO: 8 - DNA, 18 bp, synthetic construct
  source 1..18: mol_type = other DNA
tcacgttgagtggctcct

SEQ ID NO: 9 - DNA, 19 bp, synthetic construct
  source 1..19: mol_type = other DNA
agccctgaggactgtagga

SEQ ID NO: 10 - DNA, 18 bp, synthetic construct
  source 1..18: mol_type = other DNA
aacggccacttcgtttgt

SEQ ID NO: 11 - DNA, 17 bp, synthetic construct
  source 1..17: mol_type = other DNA
atctgccttccaggcca

SEQ ID NO: 12 - DNA, 17 bp, synthetic construct
  source 1..17: mol_type = other DNA
accttccactttacgct

(19) United States
(12) Patent Application Publication
(10) Pub. No.: US 2023/0190920 A1
(43) Pub. Date: Jun. 22, 2023

(54) COMPOSITIONS AND METHODS FOR LONG-LASTING GERMINAL CENTER RESPONSES TO A PRIMING IMMUNIZATION

(71) Applicants: Massachusetts Institute of Technology, Cambridge, MA (US); La Jolla Institute for Immunology, La Jolla, CA (US)

(72) Inventors: Darrell Irvine, Arlington, MA (US); Shane Crotty, San Diego, CA (US)

(21) Appl. No.: 18/047,928

(22) Filed: Oct. 19, 2022

Related U.S. Application Data
(60) Provisional application No. 63/291,433, filed on Dec. 19, 2021.

(51) Int. Cl. A61K 39/215

(52) U.S. Cl. CPC A61K 39/215; A61K 2039/545

(57) ABSTRACT

Immunization methods are provided. The methods typically include administering the subject an effective amount of an antigen and adjuvant to induce an immune response against an antigen, the method including two or more of (i) slow prime delivery of antigen and/or adjuvant, a (ii) temporally delayed 2nd immunization, and (iii) a robust adjuvant. Element (i) can be or include temporally extended exposure of antigen, adjuvant, or preferably the combination thereof, such as one or more of repeated administrations, infusion optionally by osmotic pump and escalating dosing. Element (ii) can include administering one or more boost doses of antigen and/or adjuvant, for example between 11 and 35 weeks after the start of the prime administration. A preferred robust adjuvant (iii) is one including non-liposome, non-micelle particles formed of a lipid, an additional adjuvant such as a TLR4 agonist, a sterol, and a saponin.

### CROSS-REFERENCE TO RELATED APPLICATION

This application claims the benefit of and priority to U.S. Ser. No. 63/291,433 filed Dec. 19, 2021, and which is specifically incorporated by reference herein in its entirety.

### STATEMENT REGARDING FEDERALLY SPONSORED RESEARCH OR DEVELOPMENT

This invention was made with government support under UM1 AI144462 and R01 AI125068 awarded by the National Institutes of Health. The government has certain rights in the invention.

### REFERENCE TO SEQUENCE LISTING

The Sequence Listing submitted as an .xml file named “MIT_23762_HJ_ST26.xml”, created on Oct. 12, 2022, and having a size of 11,700 bytes, is hereby incorporated by reference pursuant to 37 C.F.R. § 1.834(c)(1).

### FIELD OF THE INVENTION

This invention relates to the field of immunization technology and more specifically to compositions and administration techniques and regimens that can be used to increase immune responses against an antigen.

### BACKGROUND OF THE INVENTION

Antibodies serve as effective adaptive immunity frontline defenses against most infectious diseases. As such, most efficacious vaccines aim to prophylactically elicit potent neutralizing antibodies and long-lasting immunological memory to the target pathogen. For rapidly mutating pathogens such as HIV, there is an additional level of complication wherein an ideal vaccine should generate cross-reactive or broadly neutralizing antibodies (bnAbs) that can protect against variants (Burton & Hangartnerand, Annu. Rev. Immunol. 34, 635-659 (2016), Haynes, et al., Sci. Transl. Med. 11, (2019)) but to date bnAbs against HIV have not yet been elicited in humans nor non-human primates (NHPs) by vaccination (Lee & Crotty, Seminars in Immunology (2021) doi:10.1016/j.smim.2021.101470).

High affinity antibodies are typically the result of affinity maturation through evolutionary competition among B cells in germinal centers (GCs). GCs are evolution in miniature, with proliferation (generations) accompanied by mutations, and competition for limiting resources in the form of antigen and T cell help (Mesin, et al., Immunity 45, 471-482 (2016), Crotty, Immunity 50, 1132-1148 (2019), Victora & Nussenzweig, Annu. Rev. Immunol. 30, 429-457 (2012), Cyster & Allen, Cell 177, 524-540 (2019)). To accomplish this evolution, B_{GC} cells proliferate rapidly—every 4-6 hours (Gitlin, et al., Science (80-.). doi:10.1126/science.aac4919 (2015), Gitlin, et al., Nature 509, 637-640 (2014)). GCs are often observed for a few weeks after an acute antigen exposure. Antigen-specific B_{GC} cells have widely been observed for 14 to 28 days in most model systems, and such a time window can represent a substantial amount of antibody sequence space exploration by B_{GC} due to their fast cell cycle (Mesin, et al., Immunity 45, 471-482 (2016), Cyster & Allen, Cell 177, 524-540 (2019)). Results show that vaccine slow delivery methods over a period of 7 to 14 days, such as the use of osmotic pumps or repeated small dose injections, enhanced GC responses relative to traditional bolus immunizations in terms of magnitude of B_{GC} cells and antibody responses (Tam, et al., Proc. Natl. Acad. Sci. U.S.A. 113, E6639-E6648 (2016), Pauthner, et al., Immunity 46, 1073-1088.e6 (2017)), with some evidence of increasing the durability of GCs for two months (Cirelli, et al., Cell 177, 1153-1171.e28 (2019)). However, the full potential longevity of GCs, the biological programming of older GCs, antibody maturation under such conditions, and the functionality and productivity of older GCs are minimally understood.

Thus, it is an object of the invention to provide compositions and methods for improving antibody responses.

### SUMMARY OF THE INVENTION

Improved immunization strategies are provided. For example, methods for inducing an immune response in a subject in need thereof are provided. The methods typically include administering the subject an effective amount of an antigen and adjuvant to induce an immune response against an antigen, the method including two or more of (i) slow prime delivery of antigen and/or adjuvant, a (ii) temporally delayed 2nd immunization, and (iii) a robust adjuvant. Preferred methods include (i), (ii), and (iii).

Element (i) can be or include temporally extended exposure of antigen, adjuvant, or preferably the combination thereof. For example, the priming delivery of the antigen and/or adjuvant can include one or more of repeated administrations, infusion optionally by osmotic pump (OP), escalating dosing (ED), and sustained release carriers to increase the duration of antigen and/or adjuvant in the subject. Infusion can include, for example, continuous delivery of antigen and/or adjuvant for hours, days or weeks. In particular embodiments, the infusion is for 5-21 days inclusive, or any specific number therebetween. Continuous delivery can be of a consistent discrete dose or increasing dose of antigen and/or adjuvant. Escalating dosing can be or include administering the subject two or more doses at temporally increasing doses of antigen and/or adjuvant. The increase in dosing can be or include discrete or continuous administration, or example, 2-21 discrete administrations. Different doses can be administered, for example, hours or days apart. For example, some prime administrations include 5-10 doses, optionally 7 doses that are administered about every other day. In some embodiments, each subsequent dose is higher than the preceding dose. In some embodiments, the antigen and/or adjuvant are in a sustained release formulation. Element (i) can include administering the antigen and adjuvant in the same or different admixtures, by the same or different schedules, and administered by the same or different routes of administration. In some embodiments, element (i) includes administering the antigen and/or adjuvant by subcutaneous, intramuscular, or intravenous injection or infusion. A preferred route is subcutaneous. In particular embodiments, element (i) includes administering the antigen and adjuvant in the same admixture or different admixtures by subcutaneous administration according the same schedule.

In some embodiments, element (ii) includes administering one or more boost doses of antigen and/or adjuvant, for example between 11 and 35 weeks, or between 15 and 40 weeks, or between 20 and 35 weeks after the start or the conclusion of the prime administration of antigen and/or adjuvant. In particular embodiments, element (ii) includes administering one or more boost doses 25, 26, 27, 28, 29, 30, 32, 32, 33, 34, or 35 weeks after the start of prime dosing. In some embodiments, the booster dose includes administering the antigen and/or adjuvant by a single bolus dose or temporally extended exposure of antigen, adjuvant, or the combination thereof, optionally including any of the features of slow prime dosing. In some embodiments, element (ii) includes administering the antigen and adjuvant in the same or different admixtures, administered by the same or different schedules, by the same or different routes of administration. Routes include, but are not limited to, subcutaneous, intramuscular, or intravenous injection or infusion. In a particular embodiment, element (ii) includes administering the antigen and adjuvant in the same admixture or different admixtures by subcutaneous administration according to the same schedule.

Although other adjuvants are contemplated, a preferred robust adjuvant (iii) is one including non-liposome, non-micelle particles formed of a lipid, an additional adjuvant such as a TLR4 agonist, a sterol, and a saponin are provided. The particles are porous or perforated cage-like nanoparticles, also referred to herein as nanocages. The particles are typically between about 30 nm and about 60 nm. The particles can also include an antigen incorporated or encapsulated therein. In some embodiments, the particles do not include antigen. However, antigen, for example free, unincorporated antigen can form part of the same or a different pharmaceutical composition and be used in combination with the adjuvant particles as a vaccine.

Suitable ratios for the lipid, additional adjuvant (e.g., TLR4 agonist), sterol, and saponin components are provided. For example, in a particular embodiment, the lipid:additional adjuvant (e.g., TLR4 agonist):sterol:saponin are in a molar ratio of 2.5:1:10:10, or a variation thereof wherein the molar ratio of any one or more of the lipid, additional adjuvant, sterol, and/or saponin is increased or decreased by any value greater than 0 and up to about 3.

Exemplary lipids, additional adjuvants including TLR4 agonists, sterols, and saponins are also provided. The lipid is typically a phospholipid, such as 2-dipalmitoyl-sn-glycero-3-phosphocholine (DPPC). The sterol is most typically cholesterol or a derivative thereof. The saponin can be a natural or synthetic saponin, for example, Quil A or sub mixture or pure saponin separated therefrom. In particular embodiments, the saponin is a natural or synthetic Q-21, or an analog thereof.

Preferred additional adjuvants are TLR4 agonists. An exemplary TLR4 agonist is a lipopolysaccharide (LPS) or a lipid A derivative thereof. In particular embodiments, the lipid A derivative is a monophosphoryl lipid A such as a 4′-monophosporyl lipid A (MPLA) or 3-O-deacylated monophosphoryl lipid A (3D-MPLA).

Other additional adjuvants include, for example, pathogen-associated molecular patterns (PAMPs). In some embodiments, the PAMP is a TLR ligand, a NOD ligand, an RLR ligand, a CLR ligand, an inflammasome inducer, a STING ligand, or a combination thereof. Typically, the additional adjuvant includes a lipid to facilitate incorporation of the adjuvant into the nanocage during self-assemble. Thus, any additional adjuvant, and particularly those that do not already include one, can be modified to include a lipid.

In a specific embodiment, the lipid is DPPC, the additional adjuvant is a natural or synthetic MPLA, the sterol is cholesterol, and the saponin is Quil A or Q-21 in a molar ratio of 2.5:1:10:10.

Exemplary antigens are also provided and can be derived from, for example, a virus, bacterium, parasite, plant, protozoan, fungus, tissue or transformed cell such as a cancer. Protein (e.g., polypeptide) antigens are preferred. In particular embodiments, the antigen is derived from a coronavirus such as severe acute respiratory syndrome coronavirus 2 (SARS-CoV-2). In particular embodiments, the antigen is a spike (S) protein or a fragment or epitope(s) thereof.

In some embodiments, the antigen and/or adjuvant are administered in an effective amount and/or manner that increases GC response levels and/or duration, reduces immunodominance, and/or increases neutralizing antibodies optionally relative to traditional prime and short boost of antigen and an adjuvant consisting of alum.

### BRIEF DESCRIPTION OF THE DRAWINGS

FIGS. 1A-1D show GCs following a priming immunization can last for over half a year. FIG. 1A is an Experimental schematic. FIGS. 1B-1C are graphs showing quantification of longitudinal BGC cell kinetics. Left and right ILN FNA samples are independent data points. Triangles below indicate the prime and boost immunization time points. FIG. 1B shows quantification of total BGC cells as a percent of total CD20+B cells. FIG. 1C shows Env-binding BGC cells as a percent of total B cells. Left and right ILNs are graphed as independent data points. FIG. 1D is a series of plots showing longitudinal detection of Env-binding BGC cells in the “Long Prime” Group 3. Representative FACS plots from left ILN of one animal. Last two plots are post-boost. Gated on CD20+/CD71+CD38− BGC cells. Mean and SD or geometric mean and geometric SD are plotted depending on the scale. Limit of detection (LOD). Mann-Whitney test: *P<0.05; **P<0.005. FIG. 1E is a schematic adapted from Cirelli, et al., “Slow Delivery Immunization Enhances HIV Neutralizing Antibody and Germinal Center Responses via Modulation of Immunodominance,” Cell, 177(5):1153-1171.e28 (2019), illustrating conventional bolus dosing, continuous dosing (sustained dosage), and escalating discrete dosing.

FIGS. 2A-2H show long priming enhanced antibody quality. FIG. 2A is a line graph showing Env-binding serum IgG titers determined by ELISA. ET: Endpoint titer. FIG. 2B is a line graph showing Env-binding IgG titers following boost. Triangles indicate the boost time point. FIG. 2C is a bar graph showing BG505 pseudovirus neutralization titers at 50% inhibition (ID50). FIG. 2D is a line graph showing BG505 neutralization titers at post-boost time points. FIG. 2E is a plot showing heterologous tier-2 virus neutralization titers. ID50≤30 was considered non-neutralizing (NN). FIG. 2F is a bar graph showing the number of tier-2 heterologous viruses neutralized (ID50>50) in the 12-virus panel by week 3 post-boost serum. FIG. 2G is EMPEM and bar graph of polyclonal plasma Fabs post-boost. Group 1 at week 2 and Groups 2 & 3 at week 3 post-boost. The Env trimer is shown in gray. Graphs quantify number of animals recognizing each indicated epitope. The color of the Fabs in the EM map match that of the epitope colors in the bar graph in FIG. 2H. Fisher's exact test comparing number of epitopes recognized vs. not recognized between groups, **P<0.01. FIG. 2H is a bar graph of the epitope sites recognized by EMPEM.

FIGS. 3A-3H show BGC cell phenotypic and functional characteristics over the course of six months. FIG. 3A is a series of representative flow cytometry gating showing CD71+CD38− and Ki67+BCL6+ BGC cells. Back-gating of CD71+CD38−/Ki67+BCL6+/Env+/+ BGC cells is shown in top series. CD71−CD38+ non-BGC cells are not Ki67+BCL6+(bottom series). FIG. 3B is a plot showing the frequency of KI67+BCL6+ cells among CD71+CD38−/Env+/+ BGC. FIG. 3C is a UMAP projection of single cell gene expression profiles identifying clusters of B cell states from LN FNA and PBMCs. Int, LZ-DZ Intermediate populations. PC, Plasma cells. FIG. 3D is a series of per time point UMAP plots extracted from (FIG. 3C). FIG. 3E-3H are plot showing relative gene expression of MKI67 (FIG. 3F), AICDA (FIG. 3G), MYC (FIG. 3H), CD40 (FIG. 3I) in the DZ (DZp3) and LZ (LZ2). FIG. 3I is line graph showing DZ:LZ ratio as determine by single cell clustering in the LN after priming.

FIGS. 4A-4L show clonal competition and affinity maturation occurs in antigen-specific BGC cells identified in long lasting GCs. FIG. 4A is a line graphs showing the number of nucleotide (NT) mutations in the HC (VH+JH) of Env-binding BGC cells after priming, spaghetti plots track mutations per animal. FIG. 4B is a plot showing the number of pre- and post-boost NT mutations in Env-binding BGC cells. FIG. 4C is plot showing comparison of BGC mutations at the last pre-boost time point (pre-boost; week 10 and 29 for Group 2 & 3 respectively) and 3 weeks post-boost (week 13 and 33 for Group 2 & 3 respectively). 2-way ANOVA multiple comparisons test. FIG. 4D is line graph showing BGC population diversity at post-prime time points (Chao1). FIG. 4E is a plot showing BGC cell pre-boost population diversity. pre-boost; week 10 and 29 for Group 2 & 3 respectively. FIG. 4F is a plot showing the frequency of Env-binding BMem cells in blood. 2-Way ANOVA multiple comparisons test. FIG. 4G is a plot showing clonal diversity of BMem cells after boosting. FIG. 4H is a plot showing the number of mutations in week 6 post-boost BMem cells. FIG. 4I is a plot showing quantitation of Env-binding BMem cells that do not bind the trimer-base epitope. FIG. 4J is a plot showing serum titers of non-base directed Env-binding antibodies detected by ELISA. FIG. 4K is a plot showing clonal trees of 2 unique B cell lineages from two different long prime Group 3 animals. The tree on the left is color coded by time points while the tree on the right is color coded by Env binding. Each ring indicates 5 HC mutations from the predicted germline. For all graphs, Mann-Whitney test was used unless otherwise indicated. ns>0.05, *P<0.05, **P<0.01, ***P<0.001, ****P<0.0001. FIG. 4L is a plot showing Clonal lineage 20181, color coded by time point (top) and sample anatomical location (bottom). Clone 20181 has a 14-amino acid H-CDR3 with two or more N additions, and was represented by ten or more cells per LN. Each ring indicates five estimated HC nucleotide mutations from the most recent common ancestor.

FIGS. 5A-5C show escalating dose immunization strategy and representative flow cytometry analysis of FNA samples. FIG. 5A is a schematic showing priming ED strategy and injection schedule. FIG. 5B is a series of plots showing the gating strategy for the longitudinal analysis of GC-TFH and BGC cells from ILN FNA samples. CD71+CD38−/MD39+/+ cells were sorted for BCR sequencing and transcriptomic analyses. CD71+CD38−/MD39−/− cells were also sorted for weeks 3, 4, 7, and 10. FIG. 5C is a series of plots showing the gating strategy for the longitudinal analysis BGC cells from ILN FNA samples of Group 1 animals.

FIGS. 6A-6F show BGC and GC-TFH kinetics. FIG. 6A is a line graph showing Env-binding BGC cells as a percent of total CD20+B cells. FIG. 6B is a line graph showing Env-binding BGC cells as a percent of total BGC cells. FIG. 6C is a line graph showing kinetics of GC-TFH cells for ED/SMNP immunized animals. Gating strategy is shown in FIG. 5B. FIG. 6D is a representative flow plot of an AIM assay to detect Env-specific GC-TFH cells after 18 hrs. of ex vivo restimulation. Stimulation conditions are indicated on the right: 15-mer overlapping Env peptide megapool (Env MP), staphylococcal enterotoxin (SEB), or unstimulated (unstim.). FIG. 6E is a series of plots showing Env-specific GC-TFH cells (CXCR5hiPD-1hi, gated on FSC-A/SSC-A, FSC-H/FSC-W, SSC-H/SSC-W, Live/Dead Fixable e780−, CD4 AF700+B220 BV785−) detected at 6 weeks post boost (Group 2: week 16, Group 3: week 36) quantified by AIM assay using the indicated pair of activation markers, or positive for any two of the three AIM markers (Or gates). Data shown is background (AIM+ signal from unstimulated samples) subtracted. Left and right ILNs are graphed as independent data points. Mean and SD or geometric mean and geometric SD are plotted depending on the scale. Limit of detection (LOD). Mann-Whitney test: ns >0.05; *p<0.05. FIG. 6F is a plot showing the ratio of PE geometric mean fluorescent intensity (gMFI) of MD39+/+ B_{GC} cells to MD39neg/neg BGC cells calculated for Group 2 and 3 FNA samples at post prime time points. MD39+/+ and MD39neg/neg BGC gating is shown in FIG. 5B.

FIGS. 7A-7H show DE immunization using the SMNP adjuvant improves the quality of Env-specific antibody responses. FIG. 7A is a plot showing Group 1 autologous neutralization titers determined by assays performed by two independent labs. ID50<20 is considered non-neutralizing (NN). FIG. 7B is a plot showing Week 3 post-boost Group 2 and 3 serum neutralization ID50 titers determined independently from a second laboratory (Duke). ID50 cut-off for NN<20. †Viruses for which serum neutralization was not determined for Group 3 animal DHHT. FIG. 7C is a plot showing the number of heterologous tier-2 viruses neutralized out of 11 tested in (FIG. 7B). †Neutralization for only 6 viruses was tested for DHHT. FIGS. 7D and 7E are plots showing serum neutralization assays (Scripps) tested head-to-head with data shown in FIG. 2E using week 3 post-boost 2 sera from four RMs ED immunized with an ISCOM adjuvant (SMNP without MPLA)+BG505 (FIG. 7D) 12, and week 2 post-boost 2 sera from six RMs bolus immunized with SMNP+MD39 (FIG. 7E) (Silva, et al., Sci. Immunol. 6, (2021)). ID50 cut-off for NN<30. FIG. 7F is bar graphs showing the number of tier-2 heterologous viruses neutralized by post-boost 2 serum tested in (FIGS. 7D-7E) (Cirelli, et al., Cell 177, 1153-1171.e28 (2019), Silva, et al., Sci. Immunol. 6, (2021)). FIG. 7G is a series of plots showing number of tier-2 heterologous viruses neutralized with the indicated serum GMT titers. FIG. 7H is a plot showing the median tier-2 heterologous neutralization GMT across the 12-virus panel.

FIGS. 8A-8C show EMPEM analysis of polyclonal antibodies. FIG. 8A-8C are each a series of images showing negatively stained EM micrographs of MD39 Env trimer: polyclonal Fab complexes (top), and 2D-class averages (bottom). Data for RMs in Group 1 (FIG. 8A), Group 2 (FIG. 8B), and Group 3 (FIG. 8C). The scale bar shown in the lower left corner of each micrograph corresponds to 200 nm.

FIGS. 9A-9B showing intracellular staining of BGC markers. FIG. 9A is a chart showing some left ILN samples were pooled for the intracellular staining panel, for higher cell numbers. FIG. 9B is a representative flow plot and complete gating strategy for BCL6 and KI67 staining.

FIGS. 10A-10D show single cell transcriptional profiling of B cells. FIG. 10A is a plot showing single cell transcripts of LN FNA BGC (Group 2 and 3 weeks 3, 4, 7, 10 and 16, Group 2 week 13, Group 3 weeks 29 and 33) and PBMC BMem cells (Group 2 week 16, Group 3 week 36) were assessed. FIG. 10B is a plot showing a summary of the results from GSEA of upregulated gene profiles from single cell clusters using previously identified B cell subset gene signatures (Holmes, A. B. et al., et al. J. Exp. Med. 217, (2020)). Size of the dots represent the normalized enrichment score (NES). Significant and nonsignificant NES results are shown in different shades. FIG. 10C is a series of plot showing expression levels of the displayed genes used to help identify clusters of B cell subsets. FIG. 10D is a series of plots showing relative gene expression of MKI67, AICDA, MYC, CD40 in combined DZ and LZ clusters.

FIGS. 11A-11B show BCR sequences of BGC and BMem cells. FIGS. 11A and 11B are line graphs showing the number of NT mutations in the V and J-gene region of LC sequences (Kappa: LC κ, Lambda: LC λ) derived from Env-specific BGC cells after priming. Spaghetti plots track the number of mutations in each animal FIG. 11C is a plot showing the number of HC mutations at week 6 post-boost. FIG. 11D is series of plot showing the number of LC κ and LC λ NT mutations in BGC cells, respectively. There are two outliers among Group 3 week 3 LC κ sequences, each with 108 and 105 mutations. FIG. 11E is a gating strategy for the detection of Env-specific BMem cells. FIG. 11F is a pair of plots showing the number of LC κ and LC λ NT mutations in wk. 6 post-boost BMem cells. Mann-Whitney test, ****P<0.0001. FIG. 11G is a plot showing B_{GC} cell pre-boost clonal richness. Pre-boost; week 10 and 29 for Group 2 & 3 respectively (Chao1). FIG. 11H is a line graphs showing B_{GC} population diversity at post-prime time points (Simpson's diversity). FIG. 11I is a plot showing BGC cell pre-boost population diversity. Pre-boost; week 10 and 29 for Group 2 & 3 respectively (Simpson's diversity). FIG. 11J is a plot showing diversity of Bmem cells after boosting. Post-boost; week 16 and 36 for Group 2 & 3 respectively (Simpson's diversity).

FIGS. 12A and 12B are examples of BCR clonal lineages with increasing affinities. FIG. 12A is clonal lineages shown in FIGS. 4K, 4L, observing clones over different LN FNA and PBMC sampling periods, represented as linear phylogenic trees. MD39 Env binding KDs (M) were evaluated by surface plasmon resonance (SPR) for select monoclonal antibodies indicated by the black arrows. HC-UCA in clone 29121 refers to the HC having an identical AA sequence to the UCA. FIG. 12B is linear phylogenic trees shown in (FIG. 12A) labeled by Env-binding by flow cytometry. These phylogenies are also shown as circle plots in FIG. 4K. The grey dotted lines indicate 5 estimated HC nucleotide mutations from the most recent common ancestor.

FIGS. 13A-13E show additional examples of longitudinally assessed BCR clonal lineages. FIG. 13A shows examples of clonal lineages observed over different LN FNA and PBMC sampling periods. FIG. 13B shows B cells shown in (FIG. 13A) labeled by Env-binding based on flow cytometry. FIG. 13C shows B cells shown in (FIG. 13A) labeled according to sampling location. Clones 29431, 20181 and 10002 all have H-CDR3s >14 AA in length, contained >2 N additions and >10 cells per LN. Clone 6911 has a H-CDR3 11 AA in length, contained >2 N additions and >10 cells per LN. Each ring indicates 5 HC mutations from the nearest common ancestor. FIGS. 13D and 13E are plots showing the Pearson correlation calculated between number of total HC mutations gained from the nearest common ancestor, and time post-prime. Correlation coefficients are calculated for clones 20194 (FIG. 13D) and 21292 (FIG. 13E) shown in FIG. 4K.

FIGS. 14A-14E illustrate examples of clonal lineages with unique features. FIG. 14A shows clonal lineages where a substantial fraction of early BGC cells did not bind Env by flow cytometry. FIG. 14B shows an example of a clonal lineage that was almost exclusively IgM. In (FIG. 14A) and (FIG. 14B), each B cell is labeled according to observed time point (left), Env-binding by flow cytometry (center), and sampling location (right). FIG. 14C illustrates lineage trees shown in FIG. 4K by sampling location. Clones 21275 and 20194 have H-CDR3s >14 AA in length, contained >2 N additions and >10 cells per LN. FIG. 14D is a plot of H-CDR3 length distribution of clones found in one or both ILNs using a broader definition of clonal lineages (H-CDR3 length >10 AA, >5 cells in lineage). Lineages found in a single LN and two distal LNs exhibited similar H-CDR3 length distributions. One hypothetical concern about the definition of clonality was the possibility of two independent naïve B cells having identical H-CDR3 and L-CDR3 recombination events. If that were the driving phenomenon behind the observation of matching BGC cells in distant LNs, based on recombination event likelihoods it would be likely that length distributions would be skewed towards shorter lengths and no N-additions would be observed. FIG. 14E is a series of iNext (Hsieh, et al., Methods Ecol. Evol. 7, 1451-1456 (2016)) plots showing the results of rarefaction analysis to determine the extent of sequence coverage from the Env+/+ LN FNA and PBMC VDJ sequence data.

### DETAILED DESCRIPTION OF THE INVENTION

### I. Definitions

As used herein, “isolated,” “isolating,” “purified,” “purifying,” “enriched,” and “enriching,” when used with respect to a compound of interest, indicates that the compound of interest at some point in time were separated, enriched, sorted, etc., from or with respect to other material to yield a higher proportion of the compound of interest compared to the other materials, for example, cellular material, contaminates, or active agents such as enzymes, proteins, detergent, cations, anions, or other compounds. “Highly purified,” “highly enriched,” and “highly isolated,” when used with respect to a compound of interest, indicates that the compound of interest is at least about 70%, about 75%, about 80%, about 85%, about 90% or more, about 95%, about 99% or 99.9% or more purified or isolated from other materials such as cellular materials, contaminates, or active agents such as enzymes, proteins, detergent, cations or anions. “Substantially isolated,” “substantially purified,” and “substantially enriched,” when used with respect to a compound of interest, indicates that the compound of interest is at least about 70%, about 75%, or about 80%, more usually at least 85% or 90%, and sometimes at least 95% or more, for example, 95%, 96%, and up to 100% purified or isolated from other materials, such as cellular materials, contaminates, or active agents such as enzymes, proteins, detergent, cations or anions.

As used herein, the term “immune cell” refers to cells of the innate and acquired immune system including neutrophils, eosinophils, basophils, monocytes, macrophages, dendritic cells, lymphocytes including B cells, T cells, and natural killer cells.

As used herein, the term “immune tolerance” as used herein refers to any mechanism by which a potentially injurious immune response is prevented, suppressed, or shifted to a non-injurious immune response (Bach, et al., N. Eng. J. Med., 347:911-920 (2002)).

As used herein, the term “tolerizing vaccine” as used herein is typically an antigen-specific therapy used to attenuate autoreactive T and/or B cell responses, while leaving global immune function intact.

As used herein, the term “immunogenic agent” or “immunogen” or “antigen” is capable of inducing an immunological response against itself on administration to a mammal, optionally in conjunction with an adjuvant.

As used herein, the term “carrier” refers to an organic or inorganic ingredient, natural or synthetic, with which the active ingredient is combined to facilitate the application.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “pharmaceutically acceptable carrier” means one or more compatible solid or liquid fillers, dilutants or encapsulating substances which are suitable for administration to a human or other vertebrate animal.

As used herein, the term “treating” includes inhibiting, alleviating, preventing or eliminating one or more symptoms or side effects associated with a disease or disorder.

As used herein, the term “reduce”, “inhibit”, “alleviate” or “decrease” are used relative to a control. One of skill in the art would readily identify the appropriate control to use for each experiment.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Immunization Strategies

Long-lasting GCs have classically been observed in the context of chronic infections and gut microbiota exposure (Nowosad, et al., Nature doi:10.1038/s41586-020-2865-9 (2020), Petrovas, et al., J. Clin. Invest. doi:10.1172/JCI63039 (2012)), conditions known to have continuous live sources of renewed antigen. Recent reports of longer lasting GCs in influenza infection (Yewdell, et al., Cell Rep. 37, 109961 (2021)), SARS-CoV-2 infection (Poon, et al., Sci. Immunol. doi:10.1126/sciimmunol.ab19105 (2021)), and human RNA vaccines (Turner, et al., Nature (2021) doi:10.1038/s41586-021-03738-2, Lederer, et al., medRxiv (2021), Kim, et al., bioRxiv Prepr. Serv. Biol. (2021) doi:10.1101/2021.10.31.466651) have raised interest in the possibility of long-lasting GCs potentially under conditions of low or absent renewed antigen exposure. The experiments below demonstrate clearly that GCs can last for at least 191 days in the absence of new antigen by utilizing escalating dose (ED) protein immunization strategy and a robust adjuvant. Furthermore, the GCs were remarkably robust and functional for six months. The B_{GC} cells, maintain proliferation, SHM, and affinity maturation, and long-lasting GCs can produce high autologous tier-2 neutralizing antibody titers, heterologous neutralizing antibody titers, and highly somatically mutated circulating antigen-specific BMem cells to non-antigen base epitopes. The results inform improved immunization strategies.

The disclosed immunization strategies are based on the discovery that (i) slow delivery or antigen and/or adjuvant, a (ii) temporally delayed 2nd immunization, and (iii) a robust adjuvant can increase the strength and/or duration of a germinal center response to the antigen. Thus, the disclosed strategies typically include one, preferrable two, more preferably all three of (i)-(iii).

In some embodiments, germinal center (GC) responses remain active at least 13, 16, 21, 25, or 29 weeks after prime dosing, optionally with antigen binding to BGC cells at one of more these intervals (e.g., 29 weeks) is higher than the peak antigen binding to B_{GC} cells using conventional strategy such as alum immunization.

In some embodiments, the strategies reduce immunodominance (i.e., increase recognition of non-immunodominant epitopes) or otherwise alleviate the impact thereof, enhance neutralizing antibody development and/or breadth, or a combination thereof compared to other strategies.

A. Slow Delivery Strategies

Preferably the prime (initial) dosing of the antigen and/or adjuvant is administered using one or more slow delivery strategies. As used herein, slow delivery typically means temporally extended exposure to antigen and/or adjuvant. Preferably the slow delivery includes temporally extended exposure to both antigen and adjuvant. Typically, rather than limited to a single bolus dose of free antigen and/or adjuvant, the materials are delivered using one or more strategies including, but not limited to, repeated administrations, infusion (e.g., by osmotic pump (OP)) delivery, escalating dosing (ED), and sustained release carriers to increase the duration of antigen and/or adjuvant in the subject. Thus, the antigen and/or adjuvant can be administered to the subject together in the same admixture or separately using the same or different means of delivery occurring to the same or different schedules. Each of the antigen and adjuvant can independently have a constant or escalating dose, regardless of whether they are administered in the same or separate admixtures administered on the same or separate schedules.

For example, the subject can be administered a first dose of antigen and/or adjuvant for a first dosing period as a single discrete dose, two or more intermittent doses, or as a continuous administration (e.g., infusion); and a second dose of the composition for a second dosing period separately selected as a single discrete dose, two or more intermittent doses, or as a continuous administration (e.g., infusion), optionally followed by one or more additional doses for one or more additional dosing periods each separately selected as a single discrete dose, two or more intermittent doses, or as a continuous administration (e.g., infusion).

In some embodiments the dosage regime is a dose escalating dosage regimen. The first dose or dosing period can be a low dose (e.g., the same or loser than the next dose or dosing period). Dose escalation can be continued until a satisfactory biochemical or clinical response is reached. In some embodiments, the dosages can be maintained or steadily reduced. The methods can be used to standardize, optimize, or customize the dose level, dose frequency, or duration of the therapy.

A dosing period, which may include one or more discrete doses or continuous dosing at the same or different dosages can be minutes, hours, days, weeks or months apart. For example, in the experiments below, the escalating dosage regimen included a total dose of antigen plus adjuvant (50 μg protein, 375 μg adjuvant per side of bilateral administration) split between 7 gradually increasing doses, delivered every other day for a total of 12 days. In addition to this example, dosage, number of administrations, length of escalating administration, etc. can be varied.

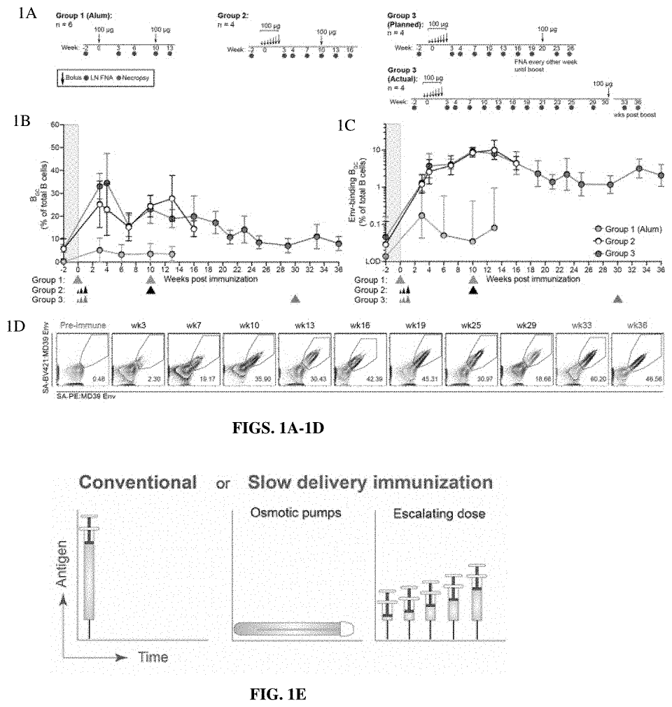

Examples of slow delivery and extended and escalating dosing are described in Tam, et al., “Sustained antigen availability during germinal center initiation enhances antibody responses to vaccination,” Proc Natl Acad Sci USA. 113(43): E6639-E6648 (2016) and Cirelli, et al., “Slow Delivery Immunization Enhances HIV Neutralizing Antibody and Germinal Center Responses via Modulation of Immunodominance,” Cell, 177(5):1153-1171.e28 (2019). doi: 10.1016/j.cell.2019.04.012, each of which is specifically incorporated by reference herein in its entirety, and exemplified in the experiments. The dosing regimens and schedules described therein can be used as expressly described therein alone or modified as further discussed elsewhere herein. For example, in Tam, et al., administering a given total dose of antigen and adjuvant over 1-2 wk. through repeated injections or osmotic pumps enhanced humoral responses, with exponentially increasing dosing profiles eliciting >10-fold increases in antibody production relative to bolus vaccination post prime. Cirelli, et al., slow delivery immunization by nonmechanical osmotic pump over two or four weeks resulted in more robust T follicular helper (T_{FH}) cell responses and GC B cells. The differences between conventional (single bolus) dosing, continuous sustained dosing, and discrete escalating dosing are illustrated in FIG. 1E, adapted from Cirelli, et al. supra, although it will be appreciated that continuous dosing and discrete escalating dosing can also be combined to achieve a continuous, escalating dosing.

Thus, exemplary prime dosage regimens include continuous or discrete administration of a consistent or escalating dose of antigen and/or adjuvant for, e.g., 7 days to 31 days inclusive, or any specific subrange or individual number therebetween, e.g., 10 days to 20 days, 12 days etc. In some embodiments, discrete dose are administered every day, every other day, every three days, every four days, every 5 days, every 6 days, or every 7 days during the dosing period.

B. Temporal Delay of Boost Immunization

The results below show that GCs can persist for greater than six months in response to a priming immunization, with a number of notable outcomes. These findings indicate that patience can have great value for allowing antibody diversification and evolution in GCs over surprisingly extended periods of time. According to the disclosed strategies, each of the antigen alone or in combination with the adjuvant can be administered as part of schedule that includes a first (“prime”) dosing period and optionally one or more (“boost”) administrations or dosing periods. Thus, in some embodiments, a boost of antigen and/or adjuvant is administered 2, 3, 4, or more times, for example, 1, 2, 3, 4, 5, 6, 7, 8, 9, 10, 11, 12, 13, 14, or 15 days, weeks, months, or years following conclusion of prime dosing. In preferred embodiments, the first boost administration of antigen and/or adjuvant is at least 10 days, at least 14 days, or between at least 3 weeks and 52 weeks inclusive after the prime administration of antigen and/or adjuvant, or any subrange or specific number of days or weeks therebetween. For example, in some embodiments, the first boost administration of antigen and/or adjuvant is between 11 and 45 weeks, or between 15 and 40 weeks, or between 20 and 35 weeks after the start or conclusion of the prime administration of antigen and/or adjuvant. The boost dosing can be a single bolus administration, or can be a slow and/or escalating dosing as discussed above. In some embodiments, no antigen and/or adjuvant is administered between the conclusion of a slow dose priming and temporarily delayed boost.

C. Strong Adjuvant

Adjuvant for use in combination with the disclosed strategies are discussed in more detail below. A preferred adjuvant is the ISCOM-type adjuvant saponin/MPLA nanoparticle (SMNP) utilized in the experiments below, and variations thereof disclosed in Silva, M. et al. “A particulate saponin/TLR agonist vaccine adjuvant alters lymph flow and modulates adaptive immunity,” Sci. Immunol. 6(66):eabf1152 (2021) doi: 10.1126/sciimmunol.abf1152., WO 2020/055503, and U.S. Published Application No. 2020/0085756 each of which are specifically incorporated by reference in their entireties. However, as discussed in more detail below, other adjuvants are also contemplated for use with the disclosed strategies.

### III. Adjuvants

Adjuvants for use in the disclosed immunization strategies and methods are provided.

A. Cage-Like Nanoparticles

1. Components of Nanocages

Cage-like nanoparticles composed of saponin, sterol, lipid, and additional adjuvant (e.g., TLR4 agonist) (also referred to as “nanocages”) are a preferred adjuvant. In some embodiments, the nanocages further include one or more antigens. In some embodiments, the nanocages do not include or incorporate antigen, but nanocage particles are present in a pharmaceutical composition with antigen (e.g., free antigen). In some embodiments, the nanocage adjuvant and antigen are part of two separate compositions. Exemplary saponins, sterols, lipids, additional adjuvants including TLR4 agonists, and antigens are discussed in more detail below.

Generally, the nanocage adjuvant is formed by mixing the components together in the presence of a detergent in a suitable ratio such that when the detergent is removed (e.g., by dialysis), the components self-assemble into nanocages. The size of the nanocages is typically dictated by the properties of the components and the self-assembly process. The disclosed compositions and methods typically yield nanocages in the range of about 30 nm and about 60 nm, or about 40 nm to about 50 nm, with a preferred size being about 40 nm.

The nanocages generally assume a distinctive porous morphology that can be structurally distinguished by transmission electronic microscope (TEM) from lipid monolayer (micelle) and lipid bilayer (liposome) particles. For example, in some embodiments, the morphological structure of the nanocages is the same or similar to the morphological structure of ISCOMATRIX®, as described and imaged in Morelli and Maraskovsky, Chapter 16—ISCOMATRIX Adjuvant in the Development of Prophylactic and Therapeutic Vaccines, Immunopotentiators in Modern Vaccines (Second Edition) 2017, Pages 311-332. Thus, preferably, the particles are not micelles or liposomes.

a. Saponin

The nanocages typically include one or more saponins. A suitable saponin is one that can induce or enhance an immune response. Saponins from plants have proven to be very effective as adjuvants. Saponins are triterpene and steroid glycosides widely distributed in the plant kingdom. Structurally, saponins are amphiphilic surfactants, which explains their surfactant properties, ability to form colloidal solutions, hemolytic activity and ability to form mixed micelles with lipids and sterols. The saponins most studied and used as adjuvants are those from Chilean tree Quillaja saponaria, which have cellular and humoral adjuvant activity. Saponins extracts from Quillaja saponaria with adjuvant activity are known and employed in commercial or experimental vaccines formulation.

A particular saponin preparation is called Quil A. Quil A is a saponin preparation isolated from the South American tree Ouillaja Saponaria Molina and was first described by Dalsgaard et al. in 1974 (“Saponin adjuvants,” Archiv. fiir die gesamte Virus forschung, Vol. 44, Springer Verlag, Berlin, p 243-254) to have adjuvant activity. The isolation of pure saponins or better defined mixtures from the Quil A product having adjuvant activity and lower toxicity than Quil A have also been described. Purified fragments of Quil A that retain adjuvant activity without the toxicity associated with Quil A (EP 0362 278), for example QS7 and QS21 (also known as QA7 and QA21), have been isolated by HPLC. QS-21 is a natural saponin derived from the bark of Quillaja Saponaria Molina, which induces CD8+ cytotoxic T cells (CTLs), Th1 cells and a predominant IgG2a antibody response. QS-21 has been used or is being studied as an adjuvant for various types of vaccines. See also EP 0 362 279 B1 and U.S. Pat. No. 5,057,540.

The isolation and adjuvant activity of other isolated Quil A saponins, including those called QS-17, and 18 have also been reported, and can also be used in the disclosed nanocages

In other embodiments, the saponin is from Quillaja brasiliensis (A. St.-Hil. et Tul.) Mart., which is native to southern Brazil and Uruguay and has saponins that have proven to be effective as adjuvants with a similar activity against viral antigens as Quil A (Silveira et al., Vaccine 29 (2011), 9177-9182).

Other useful saponins are derived from the plants Aesculus hippocastanum or Gyophila Struthium. Other saponins which have been described in the literature include escin, which has been described in the Merck index (12th ed: entry 3737) as a mixture of saponins occurring in the seed of the horse chestnut tree, Lat: Aesculus hippocastanum. Its isolation by chromatography and purification (Fiedler, Arzneimittel-Forsch. 4, 213 (1953)), and by ion exchange resins (Erbring et al., U.S. Pat. No. 3,238,190) has been described. Fractions of escin have been purified and shown to be biologically active (Yoshikawa M, et al. (Chem Pharm Bull (Tokyo) August 1996; 44(8): 1454-1464)). Sapoalbin from Gypsophila struthium (R. Vochten et al., 1968, J. Pharm. Belg., 42, 213-226) has also been described.

In other embodiments, the saponin is a synthetic saponin. See, e.g., U.S. Published Application No. 2011/0300177 and U.S. Pat. No. 8,283,456, which describe the Triterpene Saponin Synthesis Technology (TriSST) platform, a convergent synthetic approach in which the four domains in QS-21 (branched trisaccharide+triterpene+linear tetrasaccharide+fatty acyl chain) are synthesized separately and then assembled to produce the target molecule. Each of the domains can be modified independently and then combined to produce a virtually infinite number of rationally designed QS-21 analogs. Initially, fully synthetic QS-21 (SQS-21) was shown to be safe and immunologically active in a Phase 1 clinical trial, and later over 100 analogues were prepared and tested in a systematic sequential series of studies. See, e.g., Ragupathi, et al., Expert Rev Vaccines. 2011 April; 10(4): 463-470. See also Zu, et al., Journal of Carbohydrate Chemistry, Volume 33, 2014—Issue 6, pages 269-97.

Preferably the saponin component is in a substantially pure form, for example, at least 90% pure, preferably at least 95% pure and most preferably at least 98% pure.

b. Sterol

The nanocages typically include one or more sterols. Sterols include β-sitosterol, stigmasterol, ergosterol, ergocalciferol, campesterol, and cholesterol. These sterols are well known in the art, for example cholesterol is disclosed in the Merck Index, 11th Ed., page 341, as a naturally occurring sterol found in animal fat. In preferred embodiments, the sterol is cholesterol or a derivative thereof e.g., ergosterol or cholesterylhemisuccinate.

c. Lipid

The nanocages typically include one or more lipids, preferably one or more phospholipids. The lipid can be neutral, anionic, or cationic at physiologic pH. Phospholipids include, but are not limited to, diacylglycerides such as phosphatidic acid (phosphatidate) (PA), phosphatidylethanolamine (cephalin) (PE), phosphatidylcholine (lecithin) (PC), phosphatidylserine (PS), and phosphoinositides, e.g., phosphatidylinositol (PI), phosphatidylinositol phosphate (PIP), phosphatidylinositol bisphosphate (PIP2) and phosphatidylinositol trisphosphate (PIPS), as well as phosphosphingolipids such as ceramide phosphorylcholine (Sphingomyelin) (SPH), ceramide phosphoryl ethanolamine (Sphingomyelin) (Cer-PE), and ceramide phosphoryllipid, and natural and synthetic phospholipid derivatives such as egg PC (Egg lecithin), egg PG, soy PC, hydrogenated soy PC, sphingomyelin, phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), and PEG phospholipid (mPEG-phospholipid, polyglycerin-phospholipid, functionalized-phospholipid, terminal activated-phospholipid).

Thus, nanocage can include any one of more of 1,2-Didecanoyl-sn-glycero-3-phosphocholine (DDPC), 1,2-Dierucoyl-sn-glycero-3-phosphate (Sodium Salt) (DEPA-NA), 1,2-Dierucoyl-sn-glycero-3-phosphocholine (DEPC), 1,2-Dierucoyl-sn-glycero-3-phosphoethanolamine (DEPE) 1,2-Dierucoyl-sn-glycero-3[Phospho-rac-(1-glycerol) (Sodium Salt) (DEPG-NA), 1,2-Dilinoleoyl-sn-glycero-3-phosphocholine (DLOPC), 1,2-Dilauroyl-sn-glycero-3-phosphate (Sodium Salt) (DLPA-NA) 1,2-Dilauroyl-sn-glycero-3-phosphocholine (DLPC) 1,2-Dilauroyl-sn-glycero-3-phosphoethanolamine (DLPE), 1,2-Dilauroyl-sn-glycero-3[Phospho-rac-(1-glycerol) (Sodium Salt) (DLPG-NA), 1,2-Dilauroyl-sn-glycero-3[Phospho-rac-(1-glycerol) (Ammonium Salt) (DLPG-NH4), 1,2-Dilauroyl-sn-glycero-3-phosphoserine (Sodium Salt) (DLPS-NA), 1,2-Dimyristoyl-sn-glycero phosphate (Sodium Salt) (DMPA-NA), 1,2-Dimyristoyl-sn-glycero phosphocholine (DMPC), 1,2-Dimyristoyl-sn-glycero phosphoethanolamine (DMPE), 1,2-Dimyristoyl-sn-glycero-3[Phospho-rac-(1-glycerol) (Sodium Salt) (DMPG-NA), 1,2-Dimyristoyl-sn-glycero-3[Phospho-rac-(1-glycerol) (Ammonium Salt) (DMPG-NH4), 1,2-Dimyristoyl-sn-glycero-3[Phospho-rac-(1-glycerol) (Sodium/Ammonium Salt) (DMPG-NH4/NA), 1,2-Dimyristoyl-sn-glycero-3-phosphoserine (Sodium Salt) (DMPS-NA), 1,2-Dioleoyl-sn-glycero-3-phosphate (Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol) (Sodium Salt) (DOPG-NA), 1,2-Dioleoyl-sn-glycero-3-phosphoserine (Sodium Salt) (DOPS-NA), 1,2-Dipalmitoyl-sn-glycero-3-phosphate (Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol) (Sodium Salt) (DPPG-NA), 1,2-Dipalmitoyl-sn-glycero-3[Phospho-rac-(1-glycerol) (Ammonium Salt) (DPPG-NH4), 1,2-Dipalmitoyl-sn-glycero-3-phosphoserine (Sodium Salt) (DPPS-NA), 1,2-Distearoyl-sn-glycero-3-phosphate (Sodium Salt) (DSPA-NA), 1,2-Distearoyl-sn-glycero-3-phosphocholine (DSPC), 1,2-Distearoyl-sn-glycero-3-phosphoethanolamine (DSPE), 1,2-Distearoyl-sn-glycero-3[Phospho-rac-(1-glycerol) (Sodium Salt) (DSPG-NA), 1,2-Distearoyl-sn-glycero-3[Phospho-rac-(1-glycerol) (Ammonium Salt) (DSPG-NH4), 1,2-Distearoyl-sn-glycero-3-phosphoserine (Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LYSOPC MYRISTIC), 1-Palmitoyl-sn-glycero-3-phosphocholine (LYSOPC PALMITIC), 1-Stearoyl-sn-glycero-3-phosphocholine (LYSOPC STEARIC), 1-Myristoyl-2-palmitoyl-sn-glycero 3-phosphocholine (Milk Sphingomyelin MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol) . . . ] (Sodium Salt) (POPG-NA), 1-Palmitoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). Any of the lipids can be PEGylated lipids, for example PEG-DSPE.

d. Adjuvant

The nanocages optionally, but preferably, include one or more adjuvants in addition to a saponin. The additional adjuvant typically has physical and biochemical properties compatible with its incorporation into structure of the nanocage and that do not prevented nanocage self-assembly. The additional adjuvant also typically increases at least one immune response relative to the same nanocage formulation in the absence of the additional adjuvant Immune responses include, but are not limited to, an increase in an antigen-specific antibody response (e.g., IgG, IgG2a, IgG1, or a combination thereof), an increase in a response in germinal centers (e.g., increase in the frequency of germinal center B cells, an increase in frequencies and/or activation of T follicular helper (Tfh) cells, an increase in B cell presence or residence in dark zone of germinal center or a combination thereof), an increase in plasma blast frequency, an increase in inflammatory cytokine expression (e.g., IL-6, IFN-γ, IFN-α, IL-1β, TNF-α, CXCL10 (IP-10), or a combination thereof), an increase in drainage of antigen from the injection site, an in increase in antigen accumulation in the lymph nodes, an increase in lymph node permeability, an increase in lymph flow, an increase in antigen-specific B cell antigen uptake in lymph nodes, an increase in humoral responses beyond the proximal lymph node, increased diffusion of antigen into B cell follicles, or a combination thereof, when the nanocages are administered to a subject, preferably in combination with an antigen.

In preferred embodiments, the additional adjuvant is a TLR agonist. TLR4 is a transmembrane protein member of the toll-like receptor family, which belongs to the pattern recognition receptor (PRR) family Its activation leads to an intracellular signaling pathway NF-κB and inflammatory cytokine production responsible for activating the innate immune system. Classes of TLR agonists include, but are not limited to, viral proteins, polysaccharides, and a variety of endogenous proteins such as low-density lipoprotein, beta-defensins, and heat shock protein.

Exemplary TLR4 agonist include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland).

In a preferred embodiment, the TLR4 agonist is a natural or synthetic lipopolysaccharide (LPS), or a lipid A derivative thereof such as MPLA or 3D-MPLA. Lipopolysaccharides are the major surface molecule of, and occur exclusively in, the external leaflet of the outer membrane of gram-negative bacteria. LPS impede destruction of bacteria by serum complements and phagocytic cells, and are involved in adherence for colonization. LPS are a group of structurally related complex molecules of approximately 10,000 Daltons in size and contain three covalently linked regions: (i) an O-specific polysaccharide chain (O-antigen) at the outer region (ii) a core oligosaccharide central region (iii) lipid A—the innermost region which serves as the hydrophobic anchor, it includes glucosamine disaccharide units which carry long chain fatty acids.

The biological activities of LPS, such as lethal toxicity, pyrogenicity and adjuvanticity, have been shown to be related to the lipid A moiety. In contrast, immunogenicity is associated with the O-specific polysaccharide component (O-antigen). Both LPS and lipid A have long been known for their strong adjuvant effects, but the high toxicity of these molecules has precluded their use in vaccine formulations. Significant effort has therefore been made towards reducing the toxicity of LPS or lipid A while maintaining their adjuvanticity.

The Salmonella minnesota mutant R595 was isolated in 1966 from a culture of the parent (smooth) strain (Luderitz et al. 1966 Ann. N. Y. Acad. Sci. 133:349-374). The colonies selected were screened for their susceptibility to lysis by a panel of phages, and only those colonies that displayed a narrow range of sensitivity (susceptible to one or two phages only) were selected for further study. This effort led to the isolation of a deep rough mutant strain which is defective in LPS biosynthesis and referred to as S. minnesota R595.

In comparison to other LPS, those produced by the mutant S. minnesota R595 have a relatively simple structure. (i) they contain no 0-specific region—a characteristic which is responsible for the shift from the wild type smooth phenotype to the mutant rough phenotype and results in a loss of virulence (ii) the core region is very short—this characteristic increases the strain susceptibility to a variety of chemicals (iii) the lipid A moiety is highly acylated with up to 7 fatty acids.

4′-monophosporyl lipid A (MPLA), which may be obtained by the acid hydrolysis of LPS extracted from a deep rough mutant strain of gram-negative bacteria, retains the adjuvant properties of LPS while demonstrating a toxicity which is reduced by a factor of more than 1000 (as measured by lethal dose in chick embryo eggs) (Johnson et al. 1987 Rev. Infect. Dis. 9 Suppl:S512-S516). LPS is typically refluxed in mineral acid solutions of moderate strength (e.g. 0.1 M HCl) for a period of approximately 30 minutes. This process results in dephosphorylation at the 1 position, and decarbohydration at the 6′ position, yielding MPLA. In some embodiments, the TLR4 agonist is MPLA.

3-O-deacylated monophosphoryl lipid A (3D-MPLA), which can be obtained by mild alkaline hydrolysis of MPLA, has a further reduced toxicity while again maintaining adjuvanticity, see U.S. Pat. No. 4,912,094 (Ribi Immunochemicals). Alkaline hydrolysis is typically performed in organic solvent, such as a mixture of chloroform/methanol, by saturation with an aqueous solution of weak base, such as 0.5 M sodium carbonate at pH 10.5. In some embodiments, the TLR4 agonist is 3D-MPLA.

In some embodiments, the MPLA is a fully synthetic MPLA such as Phosphorylated HexaAcyl Disaccharide (PHAD®), the first fully synthetic monophosphoryl Lipid A available for use as an adjuvant in human vaccines, or Monophosphoryl 3-Deacyl Lipid A (Synthetic) (3D-PHAD®).

See also U.S. Pat. No. 9,241,988.

2. Methods of Making Nanocages

The disclosed nanocages are generally prepared by mixing together one or more saponins, one or more lipids, one or more sterols, one or more additional adjuvants (e.g., TLR4 agonist), and optionally one or more antigens in the presence of detergent. The detergent is removed, for example by dialysis. As the detergent is removed, the components self-assemble into a dispersion of nanocages. Typically the dispersion is a monodispersion. In some embodiments, the monodispersion is of particles of approximately 40 nm.

In some embodiments, one or more of the components is in an aqueous stock solution preferably including detergent and the stock solutions are then mixed together.

Preferably the detergent is a non-ionic detergent. An exemplary non-ionic detergent is Decanoyl-N-methylglucamide (MEGA-10). In some embodiments, the non-ionic detergent is about 20% of the stock solution. The solution(s) can be heated (e.g., 60-70 degrees C.) during preparation.

The components are mixed in a ratio suitable to form nanocages when the detergent is removed. In a particular embodiment, the molar ratio is 2.5:1:10:10 of Lipid:additional adjuvant (e.g., TLR4 agonist):Sterol:Saponin. The molar ratio of any component or combination thereof can be increased or decreased by any value between about 0 and about 3. Exemplary molar ratios are provided in Table 1.

TABLE 1
Exemplary Molar Ratios

| | Additional | | |
|---|---|---|---|
| Lipid | Adjuvant | Sterol | Saponin |
| 2.5 | 1 | 10 | 10 |
| 2.5 | 1 | 10 | 30 |
| 2.5 | 1 | 10 | 3 |
| 2.5 | 1 | 30 | 10 |
| 2.5 | 1 | 3 | 10 |
| 2.5 | 3 | 10 | 10 |
| 2.5 | 0.3 | 10 | 10 |
| 10 | 1 | 10 | 10 |
| 0.75 | 1 | 10 | 10 |

Thus, in some embodiments, the wherein the additional adjuvant is MPLA, the sterol is cholesterol, and the saponin is Quil A, the exemplary molar ratios can be those in Table 2.

TABLE 2
Exemplary Molar Ratios

| Lipid | MPLA | Cholesterol | Quil-A |
|---|---|---|---|
| 2.5 | 1 | 10 | 10 |
| 2.5 | 1 | 10 | 30 |
| 2.5 | 1 | 10 | 3 |
| 2.5 | 1 | 30 | 10 |
| 2.5 | 1 | 3 | 10 |
| 2.5 | 3 | 10 | 10 |
| 2.5 | 0.3 | 10 | 10 |
| 10 | 1 | 10 | 10 |
| 0.75 | 1 | 10 | 10 |

In some embodiments, the components are mixed in the following sequence: sterol, lipid, additional adjuvant (e.g., TLR4 agonist), and saponin.

In particular embodiments, the nanocages include cholesterol as the sterol, DPPC as the lipid, MPLA as the TLR4 agonist, Quil-A as the saponin, or any combination thereof. In particular embodiments, the nanocages include cholesterol as the sterol, DPPC as the lipid, MPLA as the TLR4 agonist, and Quil-A as the saponin. In even more particular embodiments, the nanocages include a molar ratio 2.5:1:10:10—DPPC:MPLA:Cholesterol:Quil-A. This embodiment is also referred to herein as “saponin-MPLA nanoparticles” and “saponin-MPLA NP.”

Once mixed, the solution can be allowed to equilibrate, for example, for 2 hours to overnight.

Next, the detergent is removed. In some embodiments, the detergent is removed by dialysis against an aqueous solution. In a particular embodiment, 10 k MWCO dialysis cassettes are used and a PBS dialysis buffer is changed about two times a day for about 4 or 5 days.

Once the detergent is removed the remaining nanocage solution can be sterile filtered using, for example, a 0.2 μm filter.

The nanocages can be purified, for example from loose components such as free additional adjuvant (e.g., TLR4 agonist), by chromatography, for example Fast Protein Liquid Chromatograph (FPLC). Suitable columns include Sephacryl S-500 HR or a similar SEC column.

Preferably, few or no liposomes or micelles are formed. However, certain preparations may yield a small fraction of worm-like micelles with a main fraction containing cage-like particles. If liposomes and/or micelles are formed during the preparation, the nanocages can be selected or separated from the liposomes and/or micelles, for example during purification.

Size/morphology can be measured by dynamic light scattering (DLS) and negative-stain TEM can be used to compare batch-to-batch homogeneity.

Antigen can be added in the presence of detergent. In such embodiments, antigen is present in solution with the other components of the nanocages, and can be incorporated into the structure of nanocages during self-assembly, when detergent is removed.

In some embodiments, antigen is not included in solution with the other components of the nanocages in the presence detergent. Antigen can be added after the detergent is removed, and thus after self-assembly is complete. In such embodiments, it is believed that the antigen will remain free and untethered or unincorporated in the nanocage.

B. Other Exemplary Adjuvants

Other adjuvants that can be used in alternative or addition to the above-discussed nanocage adjuvants, or form part of the disclosed nanocages, are also provided. Thus, in some embodiments, the adjuvant has physical and biochemical properties compatible with its incorporation into the structure of the nanocage and that do not prevented nanocage self-assembly and increase an immune response.

Suitable additional and/or alternative adjuvants include immunostimulators including those that include a lipid tail, or can be modified to contain a lipid tail. Examples of molecules that include a lipid tail, or can be modified to include one, can be, for example, pathogen-associated molecular patterns (PAMPs). PAMPS are recognized by pattern recognition receptors (PRRs). Five families of PRRs have been shown to initiate pro-inflammatory signaling pathways: Toll-like receptors (TLRs), NOD-like receptors (NLRs), RIG-I-like receptors (RLRs), C-type lectin receptors (CLRs) and cytosolic dsDNA sensors (CDSs). Also, some NLRs are involved in the formation of pro-inflammatory complexes called inflammasomes.

Thus, in some embodiments, the adjuvant is a TLR ligand, a NOD ligand, an RLR ligand, a CLR ligand, and inflammasome inducer, a STING ligand, or a combination thereof. Such ligands are known in the art can obtained through commercial vendors such as InvivoGen.

As introduced above, the ligands and other adjuvants can be modified (e.g., through chemical conjugation, for example, maleimide thiol reaction, amine N-hydroxy succinimide ester reaction, click chemistry, etc.) to include a lipid tail to facilitate incorporation of the adjuvant into the nanocage structure during self-assembly. Preferred lipids will include a 16:0 dipalmitoyl tail such as 1,2-dipalmitoyl-sn-glycero-3-phosphoethanolamine-N-[4-(p-maleimidophenyl)butyramidel]these, however, are non-limiting examples. For example, lipids of different lengths are also contemplated. In preferred embodiments, the lipid or lipids is/are unsaturated. Chemically functionalized lipids that that can be used for conjugation are known in the art and commercially available. See, for example, AVANTI® Polar Lipids, Inc. (e.g., “Headgroup Modified Lipids” and “Functionalized Lipids”).

The adjuvant can be an immunostimulatory oligonucleotide, preferable a lipidated immunostimulatory oligonucleotide. Exemplary lapidated immunostimulatory oligonucleotides and methods of making them are described in Liu, et al., Nature Letters, 507:519-22 (+11 pages of extended data) (2014)) (lipo-CpG) and U.S. Pat. No. 9,107,904, that contents of which are incorporated by reference herein in their entireties. In some embodiments, the immunostimulatory oligonucleotide portion of the adjuvant can serve as a ligand for PRRs. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76(5):2123-2129, (2008)). Therefore, the sequence of the oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun 164(3):1617-2 (2000), each of which is incorporated herein by reference).

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDAS), which are best known as RNA-sensing receptors in the cytosol. Therefore, in some embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In some embodiments, the oligonucleotide includes two or more immunostimulatory sequences.

Microbial cell-wall components such as Pam2CSK4, Pam3CSK4, and flagellin activate TLR2 and TLR5 receptors respectively and can also be used.

In other embodiments, the Addavax, Alum, ISCOMATRIX®, and ASO1B Immunostimulatory complexes called ISCOMs are particulate antigen delivery systems having antigen, cholesterol, phospholipid and saponin (Quil A or other saponin) with potent immunostimulatory activity. ISCOMATRIX® is a particulate adjuvant having cholesterol, phospholipids and saponins (Quil A) but without containing antigen. See, e.g., U.S. Pat. No. 9,149,520, Sun, et al., Volume 27, Issue 33, 16 Jul. 2009, Pages 4388-4401, and Morelli, et al., J Med Microbiol. 2012 July; 61 (Pt 7):935-43. doi: 10.1099/jmm.0.040857-0. Epub 2012 Mar. 22. This adjuvant has principally the same structure as ISCOMs, consisting of perforated cage-like particles of approximately 40 nm in diameter. The antigens can be formulated with ISCOMATRIX® to produce vaccines capable of antigen presentation and immunostimulants similar to ISCOMs-type formulations, but with a wider range of applicability, since its use is not limited to hydrophobic membrane proteins. Modifications of ISCOMs formulations and ISCOMATRIX® have also been developed to achieve a better association of some antigens, such as described in WO 98/36772.

Other liposomal systems mainly composed of saponins from Q. saponaria and sterols (primarily cholesterol) have been described, one of which is referred to as ASO1B. See, e.g., WO 96/33739, also being formulated as emulsions such as described in US 2005/0220814. See, also, U.S. Published Application No. 2011/0206758.

In preferred embodiments, the adjuvant does not consist of Alum (e.g., aluminum hydroxide, aluminum phosphate) such as Alhydrogel. Preferable the adjuvant is an ISCOM, ISCOMATRIX®, ASO1B, or most preferably a nanocage adjuvant such as those discussed above containing a TLR4 agonist, a sterol, and a saponin. See also Silva, M. et al. “A particulate saponin/TLR agonist vaccine adjuvant alters lymph flow and modulates adaptive immunity,” Sci. Immunol. 6(66):eabf1152 (2021) doi: 10.1126/sciimmunol.abf1152., WO 2020/055503, and U.S. Published Application No. 2020/0085756 each of which are specifically incorporated by reference in their entireties.

### IV. Antigen

Antigens for use in the disclosed immunization strategies and methods are provided. As discussed herein, antigen refers to the molecule to which an immune response is desired. The antigen can be a component of the adjuvant (e.g., nanocage or nanoparticulate structure itself) and/or separate and distinct therefrom (e.g., distinct from the saponin, sterol, lipid, and optional additional adjuvant (e.g., TLR4 agonist) components). Thus, in some embodiments, the adjuvant, particularly nanocage and other nanoparticulate adjuvants can optionally include, encapsulate, or incorporate one or more antigens. Such adjuvants can thus serve as both adjuvant and antigen in an immunogenic or vaccine formulation. In other embodiments, the nanocages or particulate adjuvants are absent or free of an antigen. In such embodiments, the adjuvants typically serve as an adjuvant only. In some such embodiments, antigen (e.g., free antigen) is present in a pharmaceutical composition in combination with an adjuvant that is free from/of the antigen.

Antigens can be peptides, proteins, polysaccharides, saccharides, lipids, nucleic acids, or combinations thereof. In preferred embodiments, the antigen is polypeptide. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof.

Suitable antigens are known in the art and are available from commercial, government, and scientific sources. The antigens can be whole inactivated or attenuated organisms, or derived therefrom. These organisms may be infectious organisms, such as viruses, parasites and bacteria. These organisms may also be tumor cells, or derived therefrom. For example, the antigens may be purified or partially purified polypeptides derived from tumors or viral or bacterial sources. The antigens can be recombinant polypeptides produced by expressing DNA encoding the polypeptide antigen in a heterologous expression system. The antigens can be DNA or RNA (e.g., mRNA) encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids. Exemplary sources of antigens are provided below.

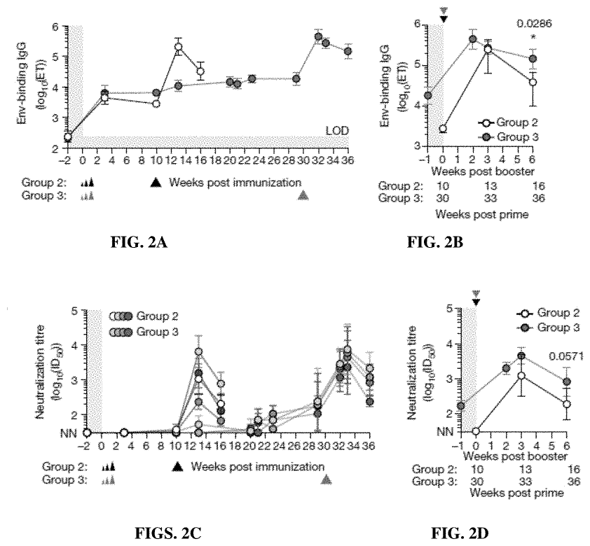

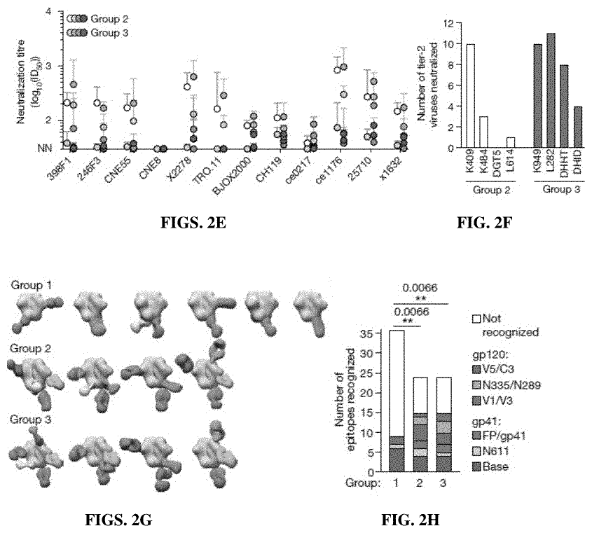

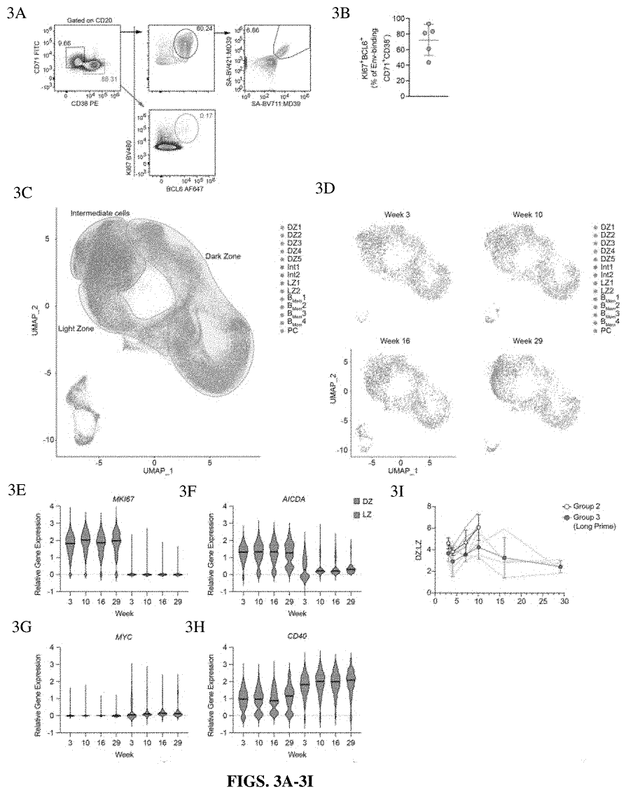

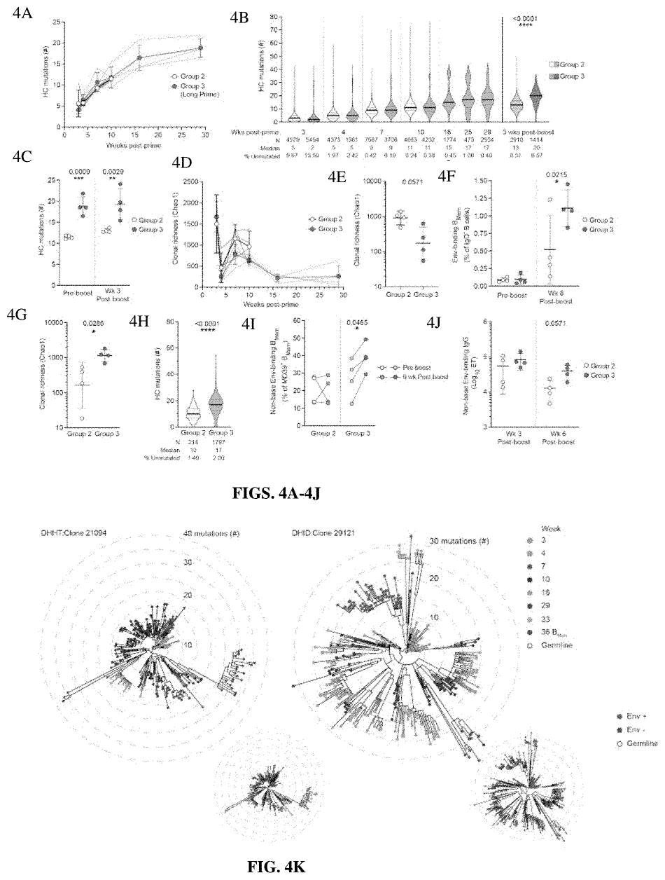

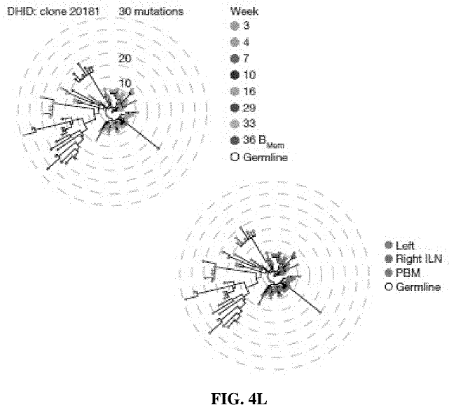

The results below show that long prime, adjuvanted, escalating dose immunization approach holds promise for difficult vaccine targets. Thus, in some embodiments, the antigen or target disease or disorder is one to which it has been difficult to mount an effective immune response against using conventional approaches. For example, in some embodiments, the antigen is one that causes immunodominance, for example when using conventional bolus dosing and/or short prime periods Immunodominance is the immunological phenomenon in which immune responses are mounted against only a few of the antigenic peptides out of the many produced. See, e.g., Akram and Inman, Clin Immunol., “Immunodominance a pivotal principle in host response to viral infections”. 2012 May; 143(2):99-115. doi: 10.1016/j.clim.2012.01.015. Results show that 12- to 14-day slow delivery (ED or osmotic pump) immunization regimen can result in substantially greater capture of vaccine antigen by stromal follicular dendritic cells (FDCs)(Cirelli, et al., Cell 177, 1153-1171.e28 (2019)). Observation of GCs for over six months indicates that endocytic recycling of immune complexes by FDCs (Heesters, et al., Immunity (2013) doi:10.1016/j.immuni.2013.02.023) can be efficient at maintaining proteins in GCs and protecting them from damage. One possible mechanism of slow delivery enhancement of GCs is improved immune complex formation, due to the supply of antigen during the earliest phases of the antibody response. Given the immunodominance of antibody responses to the non-neutralizing base of Env trimer after conventional immunizations, and epitope diversification to non-base epitopes in slow delivery immunizations (FIG. 2F), it is believed that immune complexes with Env under slow delivery conditions are primarily composed of base-binding antibodies, which shield the base of the Env trimer in GCs and orient the trimers to better display neutralizing epitopes antipodal to the base, thereby enriching for neutralizing antibody B cells. This is further illustrated by the shift away from base directed immunodominance in BMem cells during the long prime (FIG. 4I), as opposed to a normal 10-week boost that appears to recall more base-specific B cells. Thus, the improved autologous and heterologous neutralization by Group 3 animals is likely partly owing to the diversity of B cells recruited and partly due to increased affinity maturation from extensive GC responses.

A. Viral Antigens

A viral antigen can be isolated from any virus including, but not limited to, a virus from any of the following viral families Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomegalovirus, Epstein-Barr, rotavirus, rhinovirus, adenovirus, coxsackieviruses, equine encephalitis, Japanese encephalitis, yellow fever, Rift Valley fever, and lymphocytic choriomeningitis.

In some embodiments, the antigen is derived from a coronavirus. Coronavirus species and representative viruses thereof include [representative virus (of species)]: SARSr-CoV BtKY72 (Severe acute respiratory syndrome-related coronavirus), SARS-CoV-2 (Severe acute respiratory syndrome-related coronavirus), SARSr-CoV RaTG13 (Severe acute respiratory syndrome-related coronavirus), SARS-CoV PC4-227 (Severe acute respiratory syndrome-related coronavirus), SARS-CoV (Severe acute respiratory syndrome-related coronavirus), Bat-Hp-BetaCovC (Bat Hp-betacoronavirus Zhejiang2013), Ro-BatCoV GCCDC1 (Rousettus bat coronavirus GCCDC1), Ro-BatCoV HKU9 (Rousettus bat coronavirus HKU9), Ei-BatCoV C704 (Eidolon bat coronavirus C704), Pi-BatCoV HKU5 (Pipistrellus bat coronavirus HKU5), Ty-BatCoV HKU4 (Tylonycteris bar coronovirus HKU4), MERS-CoV (Middle East respiratory syndrome-related coronavirus), EriCoV (Hedgehog coronavirus), MHV (murine coronavirus), HCoV HKU1 (Human coronavirus HKU1), ChRCoV HKU24 (China Rattus coronavirus HKU24), ChRCovC HKU24 (Betacoronavirus 1), MrufCoV 2JL14 (Myodes coronavirus 2JL14), HCoV NL63 (Human coronavirus NL63), HCoV 229E (Human coronavirus 229E), and HCoV OC43 Human coronavirus OC43). See, e.g., Coronaviridae Study Group of the International Committee on Taxonomy of Viruses, Nat Microbiol 2020. DOI: 10.1038/s41564-020-0695-z), which is specifically incorporated by reference in its entirety. Common cold coronaviruses include 229E, NL63, OC43, and HKU1.

In some embodiments, the antigen is derived from a Severe acute respiratory syndrome-related virus, such as, SARSr-CoV BtKY72, SARS-CoV-2, SARSr-CoV RaTG13, SARS-CoV PC4-227, or SARS-CoV, preferably one that infects humans such as SARS-CoV or SARS-CoV-2. In some embodiments, the virus is a Middle East respiratory syndrome-related virus such as MERS-CoV.

SARS-CoV-2 coronaviruses have a genome size of approximately 26-32 kb, which encodes for the structural proteins: S, a glycoprotein that forms trimers on the viral surface and is essential for entry into the target cell; the envelope (E) protein that participates in the morphogenesis and assembly of virions, while membrane (M) and nucleocapsid (N) proteins play a fundamental role in viral RNA packaging. Sequences encoding 16 non-structural proteins (Nsp1-16) have also been identified. Antigen can be any of these proteins or a fragment or antigenic domain or epitope thereof. SARS-CoV-2 contains the Spike (S) glycoprotein on its surface, which is the main target for current vaccine development because antibodies directed against this protein can neutralize the infection. Vaccines based on the S glycoprotein, as well as its antigenic domains and epitopes, have proven effective in generating neutralizing antibodies. This, in some embodiments, the antigen is or is derived from SARS-CoV-2 spike protein. The emergence of new SARS-CoV-2 variants could affect the effectiveness of vaccines. Different types of vaccine antigens have been designed and developed against SARS-CoV-2 and can be used in combination with the disclosed compositions and methods. Examples include the complete S glycoprotein, its antigenic domains such as the receptor-binding domain (RBD) and short epitopes within the S glycoprotein. See, e.g., Martinez-Flores, et al., “SARS-CoV-2 Vaccines Based on the Spike Glycoprotein and Implications of New Viral Variants,” Front. Immunol., (12) 12 pages (2021); doi.org/10.3389/fimmu.2021.701501, which is specifically incorporated by reference herein in its entirety.

B. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

C. Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodium antigens, such as all or part of a Circumsporozoite protein, a Sporozoite surface protein, a liver stage antigen, an apical membrane associated protein, or a Merozoite surface protein.

D. Allergens and Environmental Antigens

The antigen can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including e.g., grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

E. Cancer Antigens

A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen can be MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmell 17, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

F. Neoantigens and Personalized Medicine

In some embodiments the antigen is a neoantigen or a patient-specific antigen. Recent technological improvements have made it possible to identify the immune response to patient-specific neoantigens that arise as a consequence of tumor-specific mutations, and emerging data indicate that recognition of such neoantigens is a major factor in the activity of clinical immunotherapies (Schumacher and Schreidber, Science, 348(6230):69-74 (2015). Neoantigen load provides an avenue to selectively enhance T cell reactivity against this class of antigens.

Traditionally, cancer vaccines have targeted tumor-associated antigens (TAAs) which can be expressed not only on tumor cells but in the normal tissues (Ito, et al., Cancer Neoantigens: A Promising Source of Immunogens for Cancer Immunotherapy. J Clin Cell Immunol, 6:322 (2015) doi:10.4172/2155-9899.1000322). TAAs include cancer-testis antigens and differentiation antigens, and even though self-antigens have the benefit of being useful for diverse patients, expanded T cells with the high-affinity TCR (T-cell receptor) needed to overcome the central and peripheral tolerance of the host, which would impair anti-tumor T-cell activities and increase risks of autoimmune reactions.

Thus, in some embodiments, the antigen is recognized as “non-self” by the host immune system, and preferably can bypass central tolerance in the thymus. Examples include pathogen-associated antigens, mutated growth factor receptor, mutated K-ras, or idiotype-derived antigens. Somatic mutations in tumor genes, which usually accumulate tens to hundreds of fold during neoplastic transformation, could occur in protein-coding regions. Whether missense or frameshift, every mutation has the potential to generate tumor-specific antigens. These mutant antigens can be referred to as “cancer neoantigens” Ito, et al., Cancer Neoantigens: A Promising Source of Immunogens for Cancer Immunotherapy. J Clin Cell Immunol, 6:322 (2015) doi:10.4172/2155-9899.1000322. Neoantigen-based cancer vaccines have the potential to induce more robust and specific anti-tumor T-cell responses compared with conventional shared-antigen-targeted vaccines. Recent developments in genomics and bioinformatics, including massively parallel sequencing (MPS) and epitope prediction algorithms, have provided a major breakthrough in identifying and selecting neoantigens.

Methods of identifying, selecting, and validating neoantigens are known in the art. See, for example, Ito, et al., Cancer Neoantigens: A Promising Source of Immunogens for Cancer Immunotherapy. J Clin Cell Immunol, 6:322 (2015) doi:10.4172/2155-9899.1000322, which is specifically incorporated by reference herein in its entirety. For example, as discussed in Ito, et al., a non-limiting example of identifying a neoantigen can include screening, selection, and optionally validation of candidate immunogens. First, the whole genome/exome sequence profile is screened to identify tumor-specific somatic mutations (cancer neoantigens) by MPS of tumor and normal tissues, respectively. Second, computational algorithms are used for predicting the affinity of the mutation-derived peptides with the patient's own HLA and/or TCR. The mutation-derived peptides can serve as antigens for the compositions and methods disclosed herein. Third, synthetic mutated peptides and wild-type peptides can be used to validate the immunogenicity and specificity of the identified antigens by in vitro T-cell assay or in vivo immunization.

G. Tolerogenic Antigens

The antigen can be a tolerogenic antigen. Exemplary antigens are known in the art. See, for example, U.S. Published Application No. 2014/0356384.

In some cases, the tolerogenic antigen is derived from a therapeutic agent protein to which tolerance is desired. Examples are protein drugs in their wild type, e.g., human factor VIII or factor IX, to which patients did not establish central tolerance because they were deficient in those proteins; or nonhuman protein drugs, used in a human Other examples are protein drugs that are glycosylated in nonhuman forms due to production, or engineered protein drugs, e.g., having non-native sequences that can provoke an unwanted immune response. Examples of tolerogenic antigens that are engineered therapeutic proteins not naturally found in humans including human proteins with engineered mutations, e.g., mutations to improve pharmacological characteristics. Examples of tolerogenic antigens that have nonhuman glycosylation include proteins produced in yeast or insect cells.

Tolerogenic antigens can be from proteins that are administered to humans that are deficient in the protein. Deficient means that the patient receiving the protein does not naturally produce enough of the protein. Moreover, the proteins may be proteins for which a patient is genetically deficient. Such proteins include, for example, antithrombin-III, protein C, factor VIII, factor IX, growth hormone, somatotropin, insulin, pramlintide acetate, mecasermin (IGF-1), β-gluco cerebrosidase, alglucosidase-.alpha., laronidase (α-L-iduronidase), idursuphase (iduronate-2-sulphatase), galsulphase, agalsidase-.beta. (α-galactosidase), α-1 proteinase inhibitor, and albumin.

The tolerogenic antigen can be from therapeutic antibodies and antibody-like molecules, including antibody fragments and fusion proteins with antibodies and antibody fragments. These include nonhuman (such as mouse) antibodies, chimeric antibodies, and humanized antibodies Immune responses to even humanized antibodies have been observed in humans (Getts D R, Getts M T, McCarthy D P, Chastain E M L, & Miller S D (2010), mAbs, 2(6):682-694).

The tolerogenic antigen can be from proteins that are nonhuman Examples of such proteins include adenosine deaminase, pancreatic lipase, pancreatic amylase, lactase, botulinum toxin type A, botulinum toxin type B, collagenase, hyaluronidase, papain, L-Asparaginase, rasburicase, lepirudin, streptokinase, anistreplase (anisoylated plasminogen streptokinase activator complex), antithymocyte globulin, crotalidae polyvalent immune Fab, digoxin immune serum Fab, L-arginase, and L-methionase.

Tolerogenic antigens include those from human allograft transplantation antigens. Examples of these antigens are the subunits of the various MHC class I and MHC class II haplotype proteins, and single-amino-acid polymorphisms on minor blood group antigens including RhCE, Kell, Kidd, Duffy and Ss.

The tolerogenic antigen can be a self-antigen against which a patient has developed an autoimmune response or may develop an autoimmune response. Examples are proinsulin (diabetes), collagens (rheumatoid arthritis), myelin basic protein (multiple sclerosis). For instance, Type 1 diabetes mellitus (T1D) is an autoimmune disease whereby T cells that recognize islet proteins have broken free of immune regulation and signal the immune system to destroy pancreatic tissue. Numerous protein antigens that are targets of such diabetogenic T cells have been discovered, including insulin, GAD65, chromogranin-A, among others. In the treatment or prevention of T1D, it would be useful to induce antigen-specific immune tolerance towards defined diabetogenic antigens to functionally inactivate or delete the diabetogenic T cell clones.

Tolerance and/or delay of onset or progression of autoimmune diseases may be achieved for various of the many proteins that are human autoimmune proteins, a term referring to various autoimmune diseases wherein the protein or proteins causing the disease are known or can be established by routine testing. In some embodiments, a patient is tested to identify an autoimmune protein and an antigen is created for use in a molecular fusion to create immunotolerance to the protein.

Embodiments can include an antigen, or choosing an antigen from or derived from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2.beta. (IA-213); other antigens include ICA69, ICA12 (SOX-13), carboxypeptidase H, Imogen 38, GLIMA 38, chromogranin-A, FISP-60, caboxypeptidase E, peripherin, glucose transporter 2, hepatocarcinoma-intestine-pancreas/pancreatic associated protein, S100(3, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and .alpha.-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H+, K+-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5 In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In some cases, the tolerogenic antigen is a foreign antigen against which a patient has developed an unwanted immune response. Examples are food antigens. Some embodiments include testing a patient to identify foreign antigen and creating a molecular fusion that comprises the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03 D (Mal d 1); from milk: .alpha.-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11 S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen in 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1).

Many protein drugs that are used in human and veterinary medicine induce immune responses, which create risks for the patient and limits the efficacy of the drug. This can occur with human proteins that have been engineered, with human proteins used in patients with congenital deficiencies in production of that protein, and with nonhuman proteins. It would be advantageous to tolerize a recipient to these protein drugs prior to initial administration, and it would be advantageous to tolerize a recipient to these protein drugs after initial administration and development of immune response. In patients with autoimmunity, the self-antigen(s) to which autoimmunity is developed are known. In these cases, it would be advantageous to tolerize subjects at risk prior to development of autoimmunity, and it would be advantageous to tolerize subjects at the time of or after development of biomolecular indicators of incipient autoimmunity. For example, in Type 1 diabetes mellitus, immunological indicators of autoimmunity are present before broad destruction of beta cells in the pancreas and onset of clinical disease involved in glucose homeostasis. It would be advantageous to tolerize a subject after detection of these immunological indicators prior to onset of clinical disease.

### V. Formulations

Formulations for use in the disclosed immunization strategies and methods are provided.

A. Pharmaceutical Compositions

Pharmaceutical compositions including adjuvants, antigens, and the combination thereof are provided. Pharmaceutical compositions can be for administration by parenteral (intramuscular, intraperitoneal, intravenous (IV), intradermal, or subcutaneous injection), or transmucosal (nasal, vaginal, rectal, or sublingual) routes of administration or using bioerodible inserts and can be formulated in dosage forms appropriate for each route of administration.

In some embodiments, the compositions are administered systemically, for example, by intravenous or intraperitoneal administration, in an amount effective for delivery of the compositions to targeted cells.

Most typically, the compositions are administered by intramuscular, intradermal, subcutaneous injection or infusions, or intravenous injection or infusion, or by intranasal delivery.

In certain embodiments, the compositions are administered locally, for example by injection directly into a site to be treated. In some embodiments, the compositions are injected or otherwise administered directly to one or more tumors. Typically, local injection causes an increased localized concentration of the compositions which is greater than that which can be achieved by systemic administration.

In some embodiments, the compositions are delivered by using a catheter or syringe. Other means of delivering such compositions include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the composition to the immediate area of the implant.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

An exemplary dosage range for antigen and adjuvant components of a vaccine are about 10 μg to about 500 μg of antigen and about 10 μg to about 1000 μg of adjuvant. In some embodiments, the dosage range of the antigen is between about 10 ng and about 500 μg, or about 10 ng 100 μg.

Adjuvant dosages can also be determined based on activity or units. For example, concentration of the nanocages (e.g., saponin-MPLA) can be quantified by measuring the sterol (e.g., cholesterol) content of the purified products (sigma MAK043). The sterol (e.g., cholesterol) quantification is then referred to as units of activity. In some embodiments, this value is further multiplied by the mass ratio of saponin:sterol (e.g., Quila:cholesterol) to get an estimated saponin (e.g., Quil-A) content. In some embodiments, the unit dosage of a nanocage adjuvant is between about 1 U and about 10 U, or between about 2 U and about 7 U, or between about 2.5 U and about 5 U.

1. Formulations for Parenteral Administration

In some embodiments the adjuvant, the antigen, or a combination thereof are administered in an aqueous solution, by parenteral injection.

The formulation can be in the form of a suspension or emulsion. In general, pharmaceutical compositions are provided including an effective amount of the adjuvant and optionally include pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Formulations for Topical and Mucosal Administration

The adjuvants and/or antigens can be applied topically. Topical administration can include application to the lungs (pulmonary), nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

B. Immunogenic Compositions

The adjuvants disclosed herein can be used in immunogenic compositions and as components in vaccines. Typically, immunogenic compositions disclosed herein include an adjuvant, an antigen, or a combination thereof. When administered to a subject in combination, the adjuvant and antigen can be administered in separate pharmaceutical compositions, or they can be administered together in the same pharmaceutical composition.

When present in the same pharmaceutical composition, or administered in combination, an adjuvant and an antigen can be referred to as a vaccine.

### VI. Methods of Using Immunization Strategies

Methods of using the disclosed immunization strategies and methods are provided. The disclosed compositions can be administered in an effective amount to induce, increase, or enhance an immune response. Immune response typically refers to responses that induce, increase, or perpetuate the activation or efficiency of innate or adaptive immunity.

The compositions can also be used to promote tolerance, e.g., to an allergen or autoimmune antigen.

The composition can be delivered parenterally (e.g., by subcutaneous, intradermal, or intramuscular injection) through the lymphatics, or by systemic administration through the circulatory system (e.g., by intravenous injection or infusion). In some embodiments, a adjuvant and an antigen are administered in the same manner or route. In other embodiments, the different compositions are administered in two or more different manners or routes.

In some embodiments, the compositions are delivered non-systemically. In some embodiments, at least the adjuvant alone or in combination with antigen is delivered locally. In some embodiments, the compositions are delivered by subcutaneous injection. In some embodiments, the composition is administered at a site adjacent to or leading to one or more lymph nodes which are close to the site in need of an immune response (i.e., close to a tumor or site of infection). In some embodiments, the composition is injected into the muscle. In some embodiments, the composition is administered in multiple doses at various locations throughout the body. The composition can also be administered directly to a site in need of an immune response (e.g., a tumor or site of infection).

In some embodiments, particularly those for the treatment of cancer and some infections, the adjuvant is administered without administering an antigen. It is believed that certain adjuvants such as nanocage adjuvants can still increase immune response to, for example endogenous tumor antigens or microbial antigens, without administering any further antigens to the subject.

A. Methods of Increasing an Immune Response

The immune response can be induced, increased, or enhanced by the composition compared to a control. In some embodiments, an adjuvant is administered to a subject in need thereof in an effective amount to increase an antigen-specific antibody response (e.g., IgG, IgG2a, IgG1, or a combination thereof), increase a response in germinal centers (e.g., increase the frequency of germinal center B cells, increase frequencies and/or activation T follicular helper (Tfh) cells, increase B cell presence or residence in dark zone of germinal center or a combination thereof), increase plasmablast frequency, increase inflammatory cytokine expression (e.g., IL-6, IFN-γ, IFN-α, IL-1β, TNF-α, CXCL10 (IP-10), or a combination thereof), increase drainage of antigen from the injection site, increase antigen accumulation in the lymph nodes, increase lymph node permeability, increase lymph flow, increase antigen-specific B cell antigen uptake in lymph nodes, increase a humeral response beyond the proximal lymph node, increase diffusion of antigen into B cell follicles, or a combination thereof. In some embodiments, the compositions and methods are utilized in an effective amount and/or manner that increases GC response levels and/or duration, reduces immunodominance, increases neutralizing antibodies and/or one or more other outcomes discussed herein including in the experiments below.

The control can be, for example, no adjuvant or another adjuvant. In some embodiments, the adjuvant is one that is improved relative to Alum. In some embodiments, the adjuvant is a nanocage adjuvant including an additional adjuvant such as a TLR4 agonist that can increase an immune response in a subject relative to, for example, Addavax, Alum, ISCOMATRIX®, ASO1B, or another adjuvant.

The disclosed adjuvant can be used, for example, to induce an immune response, when administering the antigen alone or in combination with an alternative adjuvant is ineffectual. In some embodiments, the nanocage adjuvant may reduce the dosage of adjuvant, antigen, or both required to induce, increase, or enhance an immune response; or reduce the time needed for the immune system to respond following administration.

Nanocage adjuvants may be administered as part of prophylactic vaccines or immunogenic compositions which confer resistance in a subject to subsequent exposure to infectious agents, or as part of therapeutic vaccines, which can be used to initiate or enhance a subject's immune response to a pre-existing antigen, such as a viral antigen in a subject infected with a virus or with cancer.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

B. Diseases to be Treated

1. Infectious Diseases

The compositions are useful for treating acute or chronic infectious diseases. Thus, the compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), coronaviruses, and common cold (e.g., human rhinovirus) viral infections. For example, pharmaceutical formulations including the composition can be administered topically to treat viral skin diseases such as herpes lesions or shingles, or genital warts. The composition can also be administered to treat systemic viral diseases, including, but not limited to, AIDS, influenza, the common cold, or encephalitis.

In humans, coronaviruses can cause respiratory tract infections that can range from mild to lethal. Mild illnesses include some cases of the common cold, while more lethal varieties can cause SARS, MERS, and COVID-19 (i.e., caused by SARS-CoV-2).

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni.

In some embodiments, the type of disease to be treated or prevented is a chronic infectious disease caused by a bacterium, virus, protozoan, helminth, or other microbial pathogen that enters intracellularly.

In particular embodiments, infections to be treated are chronic infections cause by a hepatitis virus, a human immunodeficiency virus (HIV), a human T-lymphotrophic virus (HTLV), a herpes virus, an Epstein-Barr virus, or a human papilloma virus.

2. Cancer

The compositions may be used for treating cancer, by for example, stimulating or enhancing an immune response in host against the cancer. The types of cancer that may be treated with the provided compositions and methods include, but are not limited to, the following: bladder, brain, breast, cervical, colo-rectal, esophageal, kidney, liver, lung, nasopharyngeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The compositions can be administered as an immunogenic composition or as part of vaccine, such as prophylactic vaccines, or therapeutic vaccines, which can be used to initiate or enhance a subject's immune response to a pre-existing antigen, such as a tumor antigen in a subject with cancer.

The desired outcome of a prophylactic or therapeutic immune response may vary according to the disease, according to principles well known in the art. Similarly, immune responses against cancer, may alleviate symptoms, or may be one facet in an overall therapeutic intervention against a disease. For example, administration of the composition may reduce tumor size, or slow tumor growth compared to a control. The stimulation of an immune response against a cancer may be coupled with surgical, chemotherapeutic, radiologic, hormonal and other immunologic approaches in order to affect treatment.

3. Subjects in Need of Tolerance

The compositions that increase tolerance disclosed herein can be used to inhibit immune-mediated tissue destruction for example in a setting of inflammatory responses, autoimmune and allergic diseases, and transplant rejection.

a. Inflammatory and Autoimmune Disorders

In certain embodiments, the disclosed compositions are used to treat an inflammatory response or autoimmune disorder in a subject. For example, the disclosed methods can be used to prophylactically or therapeutically inhibit, reduce, alleviate, or permanently reverse one or more symptoms of an inflammatory response or autoimmune disorder. An inflammatory response or autoimmune disorder can be inhibited or reduced in a subject by administering to the subject an effective amount of a composition in vivo, or cells modulated by the composition ex vivo.

Representative inflammatory responses and autoimmune diseases that can be inhibited or treated include, but are not limited to, rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, Guillain-Barre, Hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

b. Transplant Rejection

In another embodiment, the disclosed compositions and methods can be used prophylactically or therapeutically to reduce or inhibit graft rejection or graft verse host disease. Transplant rejection occurs when a transplanted organ or tissue is not accepted by the body of the transplant recipient. Typically rejection occurs because the immune system of the recipient attacks the transplanted organ or tissue. The disclosed methods can be used to promote immune tolerance of the transplant or graft by the receipt by administering to the subject an effective amount of a composition in vivo, or cells modulated by the composition ex vivo.

i. Transplants

The transplanted material can be cells, tissues, organs, limbs, digits or a portion of the body, for example the human body. The transplants are typically allogenic or xenogeneic. The disclosed compositions are administered to a subject in an effective amount to reduce or inhibit transplant rejection. The compositions can be administered systemically or locally by any acceptable route of administration. In some embodiments, the compositions are administered to a site of transplantation prior to, at the time of, or following transplantation. In one embodiment, compositions are administered to a site of transplantation parenterally, such as by subcutaneous injection.

In other embodiments, the compositions are administered directly to cells, tissue or organ to be transplanted ex vivo. In one embodiment, the transplant material is contacted with the compositions prior to transplantation, after transplantation, or both.

In other embodiments, the compositions are administered to immune tissues or organs, such as lymph nodes or the spleen.

The transplant material can also be treated with enzymes or other materials that remove cell surface proteins, carbohydrates, or lipids that are known or suspected of being involved with immune responses such as transplant rejection.

(a) Cells

Populations of any types of cells can be transplanted into a subject. The cells can be homogenous or heterogenous. Heterogeneous means the cell population contains more than one type of cell. Exemplary cells include progenitor cells such as stem cells and pluripotent cells which can be harvested from a donor and transplanted into a subject. The cells are optionally treated prior to transplantation as mention above.

(b) Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue. The tissue can be modified as discussed above.

(c) Organs

Exemplary organs that can be used for transplant include, but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of the composition to inhibit or reduce chronic transplant rejection relative to a control.

ii. Graft-Versus-Host Disease (GVHD)

The disclosed compositions and methods can be used to treat graft-versus-host disease (GVHD) by administering an effective amount of the composition to alleviate one or more symptoms associated with GVHD. GVHD is a major complication associated with allogeneic hematopoietic stem cell transplantation in which functional immune cells in the transplanted marrow recognize the recipient as “foreign” and mount an immunologic attack. It can also take place in a blood transfusion under certain circumstances. Symptoms of GVD include skin rash or change in skin color or texture, diarrhea, nausea, abnormal liver function, yellowing of the skin, increased susceptibility to infection, dry, irritated eyes, and sensitive or dry mouth.

In another embodiment, the disclosed compositions and methods for inducing or perpetuating a suppressive immune response can be used prophylactically or therapeutically to suppress allergies and/or asthma and/or inflammation. Allergies and/or asthma and/or inflammation can be suppressed, inhibited or reduced in a subject by administering to the subject an effective amount of a composition that promotes an immune suppressive immune response or tolerance as described above.

C. Combination Therapies

In some embodiments, the compositions are administered in further combination with one or more additional therapeutic agents. The agents can be administered in the same or separate pharmaceutical composition from the adjuvant, antigen, or combination thereof.

In some embodiments, the compositions are administered in combination with a conventional therapeutic agent used for treatment of the disease or condition being treated. Conventional therapeutics agents are known in the art and can be determined by one of skill in the art based on the disease or disorder to be treated. For example, if the disease or condition is cancer, the compositions can be co-administered with a chemotherapeutic drug; or if the disease or condition is a bacterial infection, the compositions can be co-administered with an antibiotic.

For example, when administered as a cancer vaccine, the disclosed compositions may be administered in combination with a checkpoint inhibitor (PD1, CTLA4, TIM3, etc.).

The invention can be further understood by the following numbered paragraphs:

1. A method of inducing an immune response in a subject in need thereof comprising administering the subject an effective amount of an antigen and adjuvant to induce an immune response against an antigen, the method comprising two or more of (i) slow prime delivery of antigen and/or adjuvant, a (ii) temporally delayed 2nd immunization, and (iii) a robust adjuvant.

2. The method of paragraph 1, comprising all three of (i), (ii), and (iii).

3. The method of paragraphs 1 or 2, comprising (i) wherein (i) comprises temporally extended exposure of antigen, adjuvant, or the combination thereof.

4. The method of paragraph 3, comprising (i) wherein (i) comprises temporally extended exposure to both antigen and adjuvant.

5. The method of any one of paragraphs 1-4, comprising (i) wherein (i) comprises prime delivery of the antigen and/or adjuvant using one or more of repeated administrations, infusion optionally by osmotic pump (OP), escalating dosing (ED), and sustained release carriers to increase the duration of antigen and/or adjuvant in the subject.

6. The method of paragraph 5, wherein the infusion comprises continuous delivery of antigen and/or adjuvant for hours, days or weeks.

7. The method of paragraph 6, wherein the infusion is for 5-21 days inclusive, or any specific number therebetween.

8. The method of any one of paragraphs 5-7, wherein the continuous delivery is of a consistent discrete dose or increasing dose of antigen and/or adjuvant.

9. The method of any one of paragraphs 5-8, wherein escalating dosing comprises administering the subject two or more doses at temporally increasing doses of antigen and/or adjuvant.

10. The method of paragraph 9, wherein the increase in dosing is by discrete or continuous administration.

11. The method of paragraphs 9 and 10 comprising 2-21 discrete administrations.

12. The method of any one of paragraphs 9-11, wherein different doses are administered hours or days apart.

13. The method of paragraph 12, wherein 5-10 doses, optionally 7 doses are administered about every other day.

14. The method of any one of paragraphs 9-13, wherein each subsequent dose is higher than the preceding dose.

15. The method of any one of paragraphs 5-14, wherein the antigen and/or adjuvant are in a sustained release formulation.

16. The method of any one of paragraphs 1-15, comprising (i) wherein (i) comprises administering the antigen and adjuvant in the same or different admixtures.

17. The method of paragraph 16, where the antigen and adjuvant are in different admixtures administered by the same or different schedules.

18. The method of paragraphs 16 and 17, wherein the antigen and adjuvant are administered by different routes of administration.

19. The method of any one of paragraphs 1-18, comprising (i) wherein (i) comprises administering the antigen and/or adjuvant by subcutaneous, intramuscular, or intravenous injection or infusion.

20. The method of any one of paragraphs 1-19, comprising (i) wherein (i) comprises administering the antigen and adjuvant in the same admixture or different admixtures by subcutaneous administration according to the same schedule.

21. The method of any one of paragraphs 1-20, comprising (ii) wherein (ii) comprises administering one or more boost doses is between 11 and 35 weeks, or between 15 and 40 weeks, or between 20 and 35 weeks after the start or the conclusion of the prime administration of antigen and/or adjuvant.

22. The method of any one of paragraphs 1-20, comprising (ii) wherein (ii) comprises administering one or more boost doses 25, 26, 27, 28, 29, 30, 32, 32, 33, 34, or 35 weeks after the start of prime dosing.

23. The method of any one of paragraphs 1-22, comprising (ii) wherein (ii) comprises administering the antigen and/or adjuvant by a single bolus dose or temporally extended exposure of antigen, adjuvant, or the combination thereof, optionally comprising any of the features of paragraphs 3-15.

24. The method of any one of paragraphs 1-23, comprising (ii) wherein (ii) comprises administering the antigen and adjuvant in the same or different admixtures.

25. The method of paragraph 24, where the antigen and adjuvant are in different admixtures administered by the same or different schedules.

26. The method of paragraphs 24 and 25, wherein the antigen and adjuvant are administered by different routes of administration.

27. The method of and one of paragraphs 1-26, comprising (ii) wherein (ii) comprises administering the antigen and/or adjuvant by subcutaneous, intramuscular, or intravenous injection or infusion.

28. The method of any one of paragraphs 1-27, comprising (ii) wherein (ii) comprises administering the antigen and adjuvant in the same admixture or different admixtures by subcutaneous administration according to the same schedule.

29. The method of any one of paragraphs 1-28, wherein the antigen and/or adjuvant are administered in an effective amount and/or manner that increases GC response levels and/or duration optionally relative to traditional prime and short boost of antigen and an adjuvant consisting of alum.

30. The method of any one of paragraphs 1-29, wherein the antigen and/or adjuvant are administered in effective amount and/or manner that reduces immunodominance and/or increases neutralizing antibodies optionally relative to traditional prime and short boost of antigen and an adjuvant consisting of alum.

31. The method of any one of paragraphs 1-30, comprising (iii) wherein (iii) comprises a non-liposome, non-micelle particle comprising of a lipid, a sterol, a saponin, and an additional adjuvant.

32. The method of paragraph 31, wherein the particle is a porous, cage-like nanoparticle.

33. The method of paragraph 32, wherein the porous, cage-like nanoparticle is about 30 nm to about 60 nm.

34. The method of any one of paragraphs 31-33 comprising lipid:additional adjuvant:sterol:saponin molar ratio of 2.5:1:10:10, or a variation thereof wherein the molar ratio of lipid, additional adjuvant, sterol, saponin or any combination thereof is increased or decreased by any value between about 0 and about 3.

35. The method of any one of paragraphs 31-34, wherein the lipid is phospholipid.

36. The method of paragraph 35, wherein the phospholipid is 2-Dipalmitoyl-sn-glycero-3-phosphocholine (DPPC).

37. The method of any one of paragraphs 31-36, wherein the additional adjuvant is a TLR4 agonist.

38. The method of paragraph 37, wherein the TLR4 agonist is a lipopolysaccharide (LPS) or a lipid A derivative thereof.

39. The method of paragraphs 37 or 38, wherein the TLR4 agonist is a natural or synthetic monophosphoryl lipid A (MPLA) or a derivative thereof.

40. The method of paragraph 39, wherein the MPLA or derivative thereof is natural or synthetic 4′-monophosporyl lipid A (MPLA) or 3-O-deacylated monophosphoryl lipid A (3D-MPLA).

41. The method of any one of paragraphs 1-6, wherein the additional adjuvant is a pathogen-associated molecular pattern (PAMP).

42. The method of paragraph 41, wherein the PAMP comprises a lipid.

43. The method of paragraphs 41 or 42, wherein the PAMP is a TLR ligand, a NOD ligand, an RLR ligand, a CLR ligand, an inflammasome inducer, a STING ligand, or a combination thereof.

44. The method of any one of paragraphs 41-43, wherein the additional adjuvant is a TLR4 agonist.

45. The method of any one of paragraphs 31-44 wherein the sterol is cholesterol or a derivative thereof.

46. The method of any one of paragraphs 31-45, wherein the saponin is a natural or synthetic saponin.

47. The method of paragraph 46, wherein the saponin is Quil A or submixture or pure saponin separated therefrom.

48. The method of paragraph 46, wherein the saponin a natural or synthetic Q-21, or an analog thereof.

49. The method of paragraph 31, wherein the lipid is DPPC, the additional adjuvant is a natural or synthetic MPLA, the sterol is cholesterol, and the saponin is Quil A or Q-21.

50. The method of paragraph 49, wherein the DPPC:MPLA:cholesterol:Quil A or Q-21 are in a molar ratio of 2.5:1:10:10.

51. The method of any one of paragraphs 1-50, wherein the antigen is derived from a virus, bacterium, parasite, plant, protozoan, fungus, tissue or transformed cell such as a cancer.

52. The method of paragraph 51, wherein the virus is a coronavirus.

53. The method of paragraph 52, wherein the coronavirus is a severe acute respiratory syndrome coronavirus 2 (SARS-CoV-2).

54. The method of paragraph 53, wherein the antigen comprises the spike (S) protein or a fragment or epitope(s) thereof.

55. The method of any one of paragraphs 1-54, wherein the antigen is a protein (or polypeptide or peptide) antigen.

### EXAMPLES

Germinal centers (GCs) are the engines of antibody evolution. Using HIV Env protein immunogen priming in rhesus monkeys (RM) followed by a long period without further immunization, the experiments below demonstrate GC B cells (B_{GC}) lasted at least 6 months (29 weeks), all the while maintaining rapid proliferation. A 186-fold B_{GC} cell increase was present by week 10 compared to a conventional immunization. Single cell transcriptional profiling revealed that both light zone and dark zone GC states were sustained throughout the 6 months. Antibody somatic hypermutation (SHM) of B_{GC} cells continued to accumulate throughout the 29-week priming period, with evidence of selective pressure. Additionally, Env-binding B_{GC} cells were still 49-fold above baseline 29 weeks after immunization, indicating that they could be active for significantly longer periods of time. High titers of HIV neutralizing antibodies were generated after a single booster immunization. Fully glycosylated HIV trimer protein is a complex antigen, posing significant immunodominance challenges for B cells, among other difficulties. Memory B cells (B_{Mem}) generated under these long priming conditions had higher levels of SHM, and both B_{Mem} cells and antibodies were more likely to recognize non-immunodominant epitopes. Numerous B_{GC} cell lineage phylogenies spanning the >6-month GC period were identified, demonstrating continuous GC activity and selection for at least 191 days, with no additional antigen exposure. A long prime, adjuvanted, slow delivery (12-day) immunization approach holds promise for difficult vaccine targets, and indicates that patience can have great value for tuning GCs to maximize antibody responses.

### Example 1: A Priming Immunization can Fuel GCs for Months

### Materials and Methods

Protein Expression and Purification

BG505 MD39 SOSIP Env trimers (MD39) were co-expressed with furin in HEK293F cells and expressed as previously described (Steichen, et al., Immunity 45, 483-496 (2016)). Trimers used for immunizations were expressed tag-free and quality checked for low endotoxin levels. BG505 MD39 SOSIP and BG505 MD39-base knockout (KO) trimers used as baits in flow cytometry were expressed with a C-term Avi-tag and biotinylated using a BirA biotinylation kit according to manufacturer's instructions (Avidity). The BG505 MD39-base KO trimer had the following mutations relative to the BG505 MD39 SOSIP: A73C, R500A, P561C, C605T, S613T, Q658T, L660N, A662T, and L663C.

Monoclonal antibodies were generated by cloning synthesized Fab variable-region genes into human antibody expression vectors. HCs were expressed as human IgG1. Antibody expression plasmids and recombinantly expressed monoclonal antibodies were produced by Genscript.

Animals and Immunizations

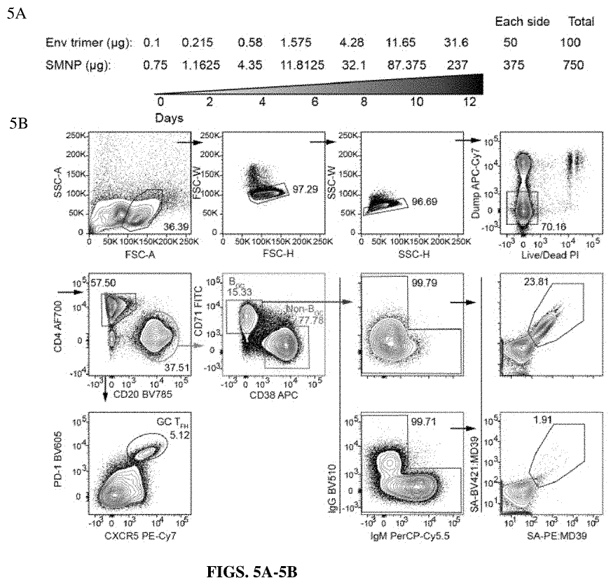

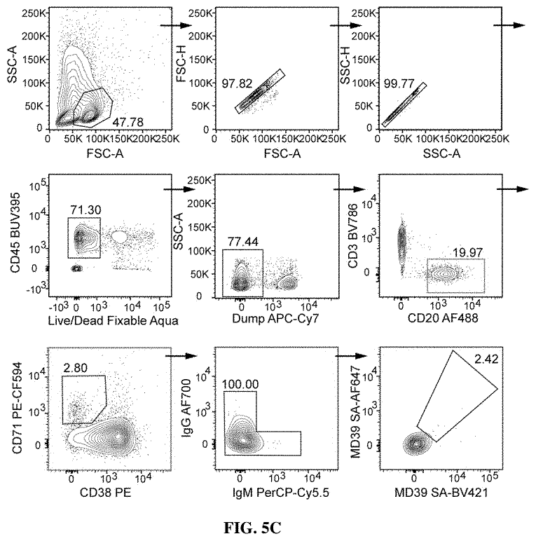

For MD39 plus SMNP ED immunization groups, Indian rhesus macaques (RMs, Macaca mulatta) were housed at Alpha Genesis Inc. and treated in accordance with protocols approved by the Alpha Genesis Inc. Animal Care and Use Committee (IACUC). 2 females and 2 males with matched aged and weight were assigned to each experimental group. Animals were 2-3 years old at the time of the priming immunization. All immunizations were given subcutaneously (s.c.) in the left and right mid-thighs with a total dose of 50 μg MD39 and 375 μg SMNP each side. For priming, a 12-day escalating dose strategy was used (FIG. 5A)(Cirelli, et al., Cell 177, 1153-1171.e28 (2019)).

For the MD39 plus alum bolus group, RMs were housed at the Tulane National Primate Research Center as part of a larger NHP study. This study was approved by the Tulane University IACUC. Animals were grouped together to match age, weight, and gender. Animals were between 3.5-5 years at time of first immunization, with 3 females and 3 males in the study group. All immunizations were given s.c. in the left and right mid-thighs with 50 μg MD39 and 500 μg alum (Alhydrogel adjuvant 2%; InvivoGen) per side. All animals were maintained in accordance with NIH guidelines.

Lymph Node Fine Needle Aspiration

LN FNAs were used to sample the left and right inguinal LNs and performed by a veterinarian. Draining lymph nodes were identified by palpation. A 22-gauge needle attached to a 3 cc syringe was passed into the LN up to 5 times. Samples were placed into RPMI containing 10% fetal bovine serum, lx penicillin/streptomycin (pen/strep). Samples were centrifuged and Ammonium-Chloride-Potassium (ACK) lysing buffer was used if the sample was contaminated with red blood cells. Samples were frozen down and kept in liquid nitrogen until analysis.

Flow Cytometry and Sorting

Frozen FNA or PBMC samples were thawed and recovered in 50% (v/v) FBS in RPMI. Recovered live cells were enumerated and stained with the appropriate staining panel. MD39 and MD39-base KO baits were prepared by mixing biotinylated MD39 with fluorophore-conjugated streptavidin (SA) in small increments at RT in an appropriate volume of 1×PBS over the course of 45 min. MD39:SA were added to the cells for 20 minutes, after which the antibody master mix was added for another 30 minutes at 4° C. Where KO baits were used, KO baits were added to the cells first for 20 minutes, then WT MD39:SA baits and were added for another 20 min, followed by the addition of the remainder of the staining panel for an additional 30 minutes at 4° C., similar to previously described¹². Fully supplemented RPMI (R10; 10% (v/v) FBS, lx pen/strep, lx Glutamax) was used as FACS buffer. For sorting, anti-human hashtag antibodies (Biolegend) were individually added to each sample at a concentration of 2.5 μg/5 million cells at the time of addition of the master mix. Group 1 samples were sorted on a FACSFusion (BD Biosciences) and Group 2 and 3 samples were either acquired or sorted on a FACSymphony S6 (BD Biosciences). Indexed V(D)J, Feature Barcode, and GEX libraries of sorted samples were prepared according to the protocol for Single Indexed 10X Genomics V(D)J with Feature barcoding kit (10X Genomics). Custom primers were designed to target RM BCR constant regions. Primer set for PCR 1; forward: AATGATACGGCGACCACCGAGATCTACACTCTTTCCCTACACGAC GCTC (SEQ ID NO:1), reverse: AGGGCACAGCCACATCCT (SEQ ID NO:2), TTGGTGTTGCTGGGCTT (SEQ ID NO:3), TGACGTCCTTGGAAGCCA (SEQ ID NO:4), TGTGGGACTTCCACTGGT (SEQ ID NO:5), TGACTTCGCAGGCATAGA (SEQ ID NO:6). Primer set for PCR 2; forward: AATGATACGGCGACCACCGAGATCT (SEQ ID NO:7), reverse: TCACGTTGAGTGGCTCCT (SEQ ID NO:8), AGCCCTGAGGACTGTAGGA (SEQ ID NO:9), AACGGCCACTTCGTTTGT (SEQ ID NO:10), ATCTGCCTTCCAGGCCA (SEQ ID NO:11), ACCTTCCACTTTACGCT (SEQ ID NO:12). Forward primers were used at a final concentration of 1 μM and reverse primers at 0.5 μM each per 100 uL PCR reaction. Libraries were pooled and sequenced on a NovaSeq Sequencer (Illumina) as previously described (Lee, et al., npj Vaccines (2021) doi:10.1038/s41541-021-00376-7).

During the long prime tracking phase (weeks 16-25, right LNs), samples were stained as described above, fixed in BD Cytofix (BD Biosciences), then analyzed on a FACSCelesta (BD Biosciences). For intracellular staining, cells were stained as described above then fixed with Foxp3/Transcription factor staining kit (Invitrogen). Cells were washed with 1× diluted permeabilization buffer then stained for 1 hr with antibodies targeting transcription factors of interest. Cells were washed and analyzed on a Cytek Aurora (Cytek Biosciences). All flow cytometry data were analyzed in Flowjo v10 (BD Biosciences).

For LN FNA data inclusion for GC gating for MD39 plus alum (Group 1), a threshold of 250 total B cells in the sample was used. For Env-binding GC B cell gating, a threshold of 75 total GC B cells was used. Any sample with fewer than 75 GC B cells but with a B cell count of >500 cells was set to a baseline of 0.001% Env⁺ GC B cells (% of B). Otherwise the limit of detection was calculated based on the median of [3/(number of B cells collected)] from the pre-immunization LN FNA samples.

The following reagents were used for staining: Alexa Fluor 647 SA (Invitrogen), BV421 SA (Biolegend), BV711 SA (Biolegend), PE SA (Invitrogen), Live/Dead fixable aqua (Invitrogen), Propidium iodide (Invitrogen), eBioscience Fixable Viability Dye eFluor 780 (Invitrogen), mouse anti-human CD20 BV785, BUV395, Alexa Fluor 488, PerCP-Cy5.5 (2H7, Biolegend), mouse anti-human IgM PerCP-Cy5.5, BV605 (G20-127, BD Biosciences), mouse anti-human CD4 BV650, Alexa Fluor 700 (OKT4, Biolegend), mouse anti-human PD1 BV605 (EH12.2H7, Biolegend), mouse anti-human CD3 BV786, APC-Cy7 (Sp34-2, BD Biosciences), mouse anti-human CXCR5 PE-Cy7 (MUSUBEE, ThermoFisher), mouse anti-human CD71 PE-CF594 and FITC (L01.1), mouse anti-human CD38 PE, APC (OKT10, NHP Reagents), mouse anti-human CD8a APC eFluor 780 (RPA-T8, ThermoFisher), mouse anti-human CD14 APC-Cy7 (M5E2, Biolegend), mouse anti-human CD16 APC-Cy7 (3G8, Biolegend), mouse anti-human CD16 APC-eFluor 780 (ebioCD16, Invitrogen), mouse anti-human IgG Alexa Fluor 700, BV510, and BV786 (G18-145, BD Biosciences), Mouse anti-NHP CD45 BUV395 (D058-1283, BD Biosciences), mouse anti-human BCL6 Alexa Fluor 647 (K112-91, BD Biosciences), mouse anti-human KI67 BV480 (B56, BD Biosciences), mouse anti-human FoxP3 BB700 (236A/E7, BD Biosciences), mouse anti-human CD27 PE-Cy7 (0323, Biolegend), goat anti-human IgD FITC (polyclonal, Southern Biotech), Armenian hamster anti-mouse/human Helios PE/Dazzle 594 (22F6, Biolegend), TotalSeq-C anti-human Hashtag antibody 1-8 (LNH-94 and 2M2, Biolegend), TotalSeq-00953 PE Streptavidin (Biolegend).

Detection of Antigen-Specific GC-T_{FH} Cells

Antigen induced marker (AIM)-based identification of Env-specific GC-T_{FH} cells was performed as previously described (Cirelli, et al., Cell 177, 1153-1171.e28 (2019), Havenar-Daughton, et al., J. Immunol. 197, 994-1002 (2016)). In summary, cells were thawed in 50% (v/v) FBS in RPMI and resuspended in 500 μL of DNase in R10 (100 μL DNAse in 900 μL R10) for 15 min at 37° C. in a CO₂ and humidity controlled incubator. 5 mL R10 was added and cells were further rested for 3 hrs. Cells were enumerated and seeded at ˜1 million cells per well in R10, and incubated with a final concentration of 2.5 μg/mL MD39 Env peptide pool, 10 pg/mL SEB, or media only (unstimulated) for 18 hrs. at 37° C. in a CO₂ and humidity controlled incubator. 1:100 mouse anti-human CXCR5 PE-Cy7 (MUSUBEE, ThermoFisher) was added to each well at the start of stimulation. Cells were washed and stained for 45 min in the dark at 4° C. After staining, cells were washed and fixed with BD Cytofix (BD Biosciences) and analyzed on a BD FACSCelesta (BD Biosciences). The following antibodies were used in the flow panel: mouse anti-human CD4 Alexa Fluor 700 (OKT4, Biolegend), mouse anti-human CD20 BV785 (2H7, Biolegend), mouse anti-human PD1 BV605 (EH12.2H7, Biolegend), mouse anti-human CXCR5 PE-Cy7 (MUSUBEE, ThermoFisher), mouse anti-human CD134 PE (L106, BD Biolegend), mouse anti-human 4-1BB APC (4B4-1, Biolegend), mouse anti-human CD25 FITC (BC96, Biolegend), mouse anti-human CD16 APC-eFluor 780 (ebioCD16, Invitrogen), mouse anti-human CD8a APC eFluor 780 (RPA-T8, ThermoFisher), mouse anti-human CD14 APC-Cy7 (M5E2, Biolegend), eBioscience Fixable Viability Dye eFluor 780 (Invitrogen).

Graphs, Statistics, and Cell Generation Calculation

All statistics were calculated in Prism 9 or R. The statistical tests used are indicated in the respective figure legends and utilize a two-tailed test. All graphs were generated in Prism 9 or R. Geometric mean and geometric standard deviation (SD) are shown for data plotted on a Logic, axis. Mean and SD are plotted for data graphed on a linear axis. Median and quartiles 1 and 3 are shown for violin plots. UMAP plots were generated using Seurat V4 (Hao, Cell doi:10.1016/j.cell.2021.04.048 (2021)). For comparison of total HC and LC mutations between Group 2 and Group 3, statistical significance was calculated only if mean mutations were significantly different by per animal comparisons.

Diversity Analysis

Chao1 estimation of clonal richness was calculated according to the following formula:

$${S}_{Chao 1} = {S}_{obs} + \frac{{F}_{1} ( {F}_{1} - 1 )}{2 ( {F}_{2} + 1 )}$$

Where S_{obs} is the total number of observed species and F₁ and F₂ are the number of singletons and doubletons, respectively. Simpson diversity indices were computed using the alphaDiversity function from Alakazam, with uniform resampling down to 50 for LN FNA samples and 25 for PBMC samples to correct for sequencing depth, at diversity order (q)=2. FNA samples containing fewer than 50 cells and PBMC samples containing fewer than 25 cells were excluded from diversity analysis. Simpson diversity (D) was calculated using the inverse Simpson index formula where R is richness (the total number of unique lineages in sample) and P, is the proportional abundance of the ith lineage:

$${D}_{2} = \frac{1}{{\sum }_{i = 1}^{R} {P}_{i}^{2}}$$

### Results

Alum is a classic adjuvant used in many human vaccines (Cirelli, et al., Cell 177, 1153-1171.e28 (2019)). A group of rhesus monkeys (RMs) were given bolus injections of recombinantly expressed stabilized HIV Env trimer (Steichen, et al., Immunity 45, 483-496 (2016)), MD39 (50 μg protein) formulated with alum adjuvant (Alhydrogel), reflective of how most licensed human protein vaccines are formulated and administered (Group 1, FIG. 1A). In an effort to generate more robust GCs, two groups of RMs were immunized with MD39 Env trimer formulated with the new ISCOM-type adjuvant saponin/MPLA nanoparticle (SMNP) (Silva, et al., Sci. Immunol. 6, (2021)) (Groups 2 & 3, FIG. 1A). The priming immunization for these two groups was administered via a slow delivery vaccination method termed escalating dose (ED) (Tam, et al., Proc. Natl. Acad. Sci. U.S.A. 113, E6639-E6648 (2016)), where the total dose of the MD39 plus SMNP formulation (50 μg protein, 375 μg adjuvant per side) was split between 7 gradually increasing doses, delivered every other day for a total of 12 days (FIG. 5A). Group 3 was designed with an unusual “long prime” period to assess the durability of GCs after a primary immunization. Each animal in the study was immunized bilaterally, thereby doubling the number of lymph nodes (LNs) and GCs that could be tracked over time. GCs were sampled every 2-3 weeks by LN fine needle aspiration (FNA) of inguinal LNs (ILNs) (FIG. 1A).

Following the priming immunization, conventional MD39 plus alum-bolus immunized animals (Group 1) exhibited an increase in total B_{GC} cell (CD71⁺CD38⁻) % at week 3 post-immunization (FIG. 1B); the frequency of Env-binding B_{GC} cells (CD71⁺CD38⁻/Env^{+/+}) peaked at week 3 and declined thereafter (Env-binding B_{GC} cells as % of total B cells. FIG. 1C). Both total and Env-binding B_{GC} cells were substantially increased in MD39 plus SMNP ED—immunized RMs compared to RMs that received conventional protein plus alum bolus immunization (Groups 2 & 3 combined vs. Group 1, FIG. 1B-1C, FIG. 5B, 6A). Median peak B_{GC} cell frequencies observed were 24-33% compared to 3.5% (Mann-Whitney P<0.0001, week 3 Groups 2 & 3 combined vs. Group 1. FIG. 1B). Median Env-binding BGC cell frequencies were approximately 7.8-times greater at week 3 (Mann-Whitney, P<0.0025, Groups 2 & 3 combined vs Group 1. FIG. 1C). Strikingly, in contrast to the conventionally primed Group 1, frequencies of Env-binding B_{GC} cells in Groups 2 and 3 continued to increase, resulting in a 186-fold GC difference by week 10 (Mann-Whitney, P<0.0001, Group 2 & 3 combined vs Group 1. (FIG. 1C)).

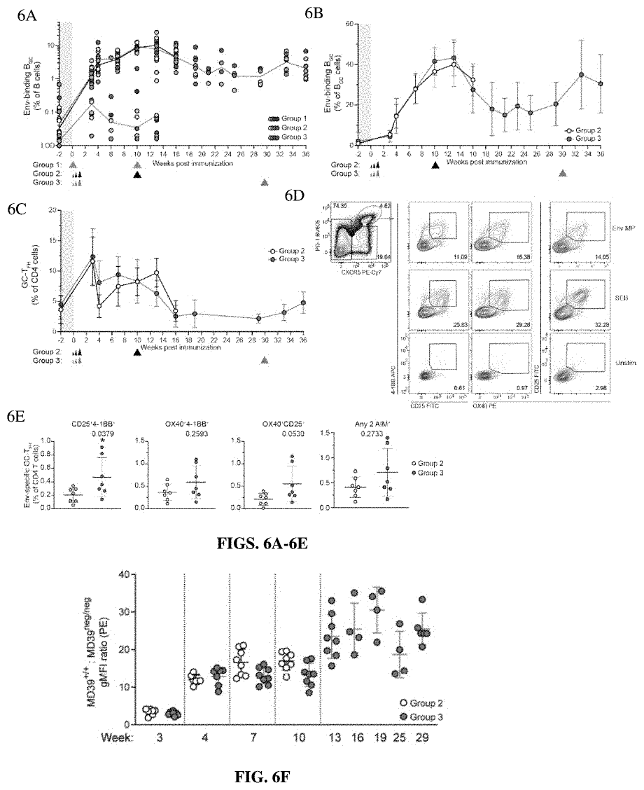

Tracking of the priming immune response continued for Group 3 animals beyond week 10 (Group 1 & 2 animals were boosted at week 10, FIG. 1A). GC responses were still active at weeks 13, 16, 21, 25, and 29 (FIGS. 1B-1D, FIGS. 6A-6B). The median magnitude of these Env-binding B_{GC} cells at week 29 was still 27-fold higher than the peak Env-binding B_{GC} cells observed after conventional alum immunization, and it was also greater than the post-boost Env-binding B_{GC} cell response to conventional alum immunization (FIG. 1C). 191 days (27 weeks) after the end of the priming dose (29 weeks from day 0), median Env-binding B_{GC} cell frequencies in ILNs were still ˜49-fold higher than baseline (FIGS. 1B-1D, FIGS. 6A-6B). Thus, GCs were capable of continuous activity for >191 days with no additional antigen introduced.

GC-T follicular helper (T_{FH}) cells play a critical role in the recruitment and selection of B_{GC} cells (Crotty, Immunity 50, 1132-1148 (2019)). Total GC-T_{FH} cell frequencies in ILNs changed over the course of the priming period (FIG. 6C), however longitudinal quantitation of Env-specific GC-T_{FH} cells was not possible due to limiting FNA samples. At 6 weeks post-boost, increased Env-specific GC-T_{FH} cell frequencies trended higher in the long prime Group 3 (FIGS. 6D-6E) (Havenar-Daughton, et al., J. Immunol. 197, 994-1002 (2016)). Long-lasting prime GCs may contribute to an improved antigen-specific GC-T_{FH} response after the booster immunization.

### Example 2: Enhanced Antibody Response Quality

### Materials and Methods

Neutralization Assays

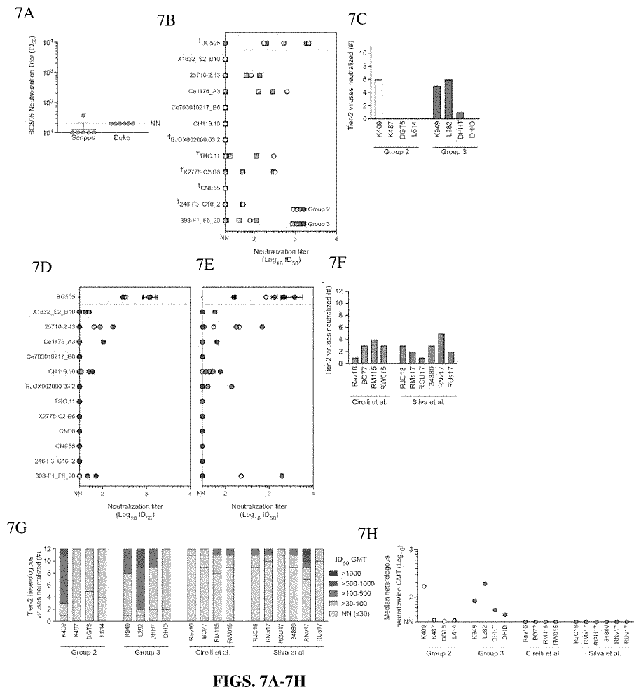

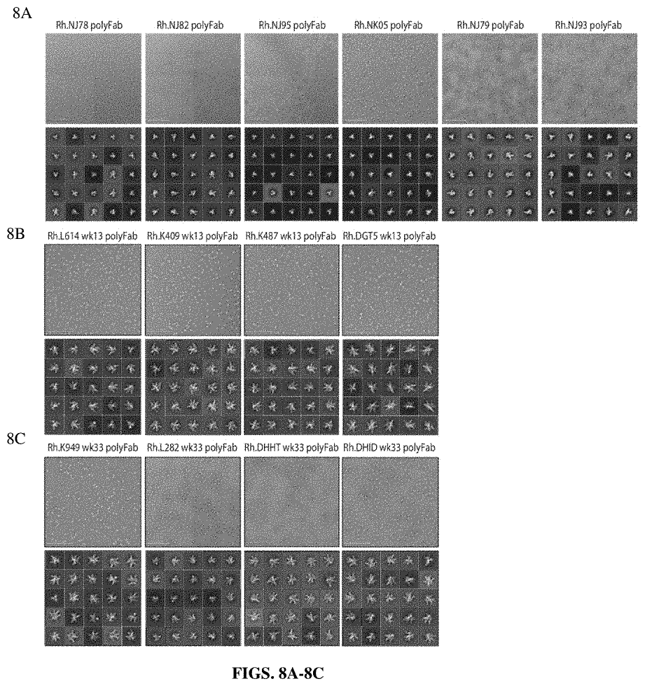

Pseudovirus neutralization assays were performed as previously described (Pauthner, et al., Immunity 46, 1073-1088.e6 (2017)). BG505 pseudovirus neutralization was tested using the BG505.W6M.ENV.C2 isolate with the T332N mutation to restore the N332 glycosylation site, except in FIGS. 7A and 7B, where the original T332 strain was used. Heterologous neutralization breadth was tested on a panel of 12 cross-clade isolates, representative of larger virus panels isolated from diverse geography and clades (deCamp, et al., J. Virol.doi:10.1128/jvi.02853-13 (2014)). The cut-off for neutralizing serum dilution was set at 1:30 or 1:20 depending on the starting serum dilution. Absolute ID₅₀s were calculated using normalized RLU values and a customized non-linear regression model:

$${ID}_{50} = Bottom + \frac{Top - bottom}{1 + {10}^{( log ( absolute 1 {C}_{50} - x ) \times Hill slope + log ( \frac{top - bottom}{50 - bottom} - 1 )}}$$

with the bottom constraint (Bottom) set to 0 and top constraint (Top) set to <100 model in Prism 8 (GraphPad). Pseudovirus neutralization assays at Duke were performed as previously described (Sarzotti-Kelsoe, et al., Immunol. Methods 409, 131-146 (2014)). Positive controls (monoclonal antibodies) were included for every virus in every assay run and tracked as part of assay quality control, as well as a murine leukemia virus negative control. Duke samples were assayed in duplicate and assays were performed with good clinical laboratory practice compliance.

ELISA

Plasma samples were thawed, heat inactivated at 56° C. for at least 30 min, and spun down. 96-well half area plates (Corning) were coated overnight with SA at 2.5 μg/mL. Plates were washed 3× with wash buffer (PBS, 0.05% (v/v) Tween-20), then coated with biotinylated MD39 or MD39-base KO trimers at 1 μg/mL. After washing 3× with wash buffer, plates were blocked with blocking buffer (PBS, 3% (w/v) BSA) for 1 hr at RT. Plasma serially diluted in blocking buffer were allowed to bind the trimers for 1 hr at RT. Plates were washed 3× and incubated with goat anti-rhesus IgG-HRP antibody (Southern Biotech, 1:10,000 in blocking buffer) for 1 hr at RT. Plates were washed 6×, and developed with 1-Step Ultra TMB (ThermoFisher). The reaction was stopped with an equivalent volume of 2N H₂SO₄ (Ricca Chemical Company) and signal was read at OD 450 nm on an EnVision plate reader (Perkin Elmer). Endpoint titers were interpolated from a Asymmetric Sigmoidal, 5PL X is log(concentration) model in Prism 9 (GraphPad).

EMPEM Analysis

Polyclonal EM analysis was performed as previously described (Bianchi, et al., Immunity 49, 288-300.e8 (2018), Nogal, et al., Cell Rep. 30, 3755-3765.e7 (2020)). Plasma antibodies were purified using Protein A Sepharose resin (GE Healthcare), eluted from the resin with 0.1 M glycine at pH 2.5 and buffer exchanged into 1×PBS. Fabs were generated using crystalline papain (Thermo Scientific) and digested for 5 h at 37° C., and purified via size exclusion chromatography (SEC) using Superdex 200 Increase 10/300 column (GE Healthcare). Complexes were assembled with 0.5 mg of polyclonal Fabs incubated overnight with 15 μg of MD39 Env trimers at RT, followed by purification to remove unbound Fab via size exclusion chromatography (SEC) using a Superose 6 Increase 10/300 column (GE Healthcare). Complexes were diluted to 30-50 μg/mL and immediately placed on 400-mesh Cu grids and stained with 2% (w/v) uranyl formate for 40 s. Images were collected via the Leginon automated imaging interface using either a Tecnai Spirit electron microscope, operating at 120 kV, or a Tecnai TF20 electron microscope operated at 200 kV. For the Spirit, nominal magnification was 52,000×, with a pixel size of 2.06 Å. The TF20 was operated at a nominal magnification of 62,000× with a pixel size of 1.77 Å for the TF20. Micrographs were recorded using a Tietz 4 k×4 k TemCam-F416 CMOS camera. Particles were extracted via the Appion data processing package³⁵ where approximately 100,000 particles were auto-picked and extracted. Using Relion 3.0 (Zivanov, et al., Elife 7, (2018)), particles were 2D-classified into 100 classes and particles with antigen-Fab characteristics were selected for 3D analysis. Initially, 3D classification was done using 20-40 classes, with a low-resolution model of a non-liganded HIV Env ectodomain used as a reference. Particles from similar looking classes were combined and reclassified, with a subgroup of 3D classes processed using 3D auto-refinement. UCSF Chimera 1.13 was used to visualize and segment the 3D refined maps.

Quantitation of epitope recognition in FIG. 2H was done by tabulating the number of epitopes recognized among each animal per group and then collation per group (for example, out of a total of six epitope sites×4 animals=24). Epitope sites not recognized per animal were also tabulated, to account for variation in animal group size. Fisher's exact tests were used to test for significant differences in the number of epitopes recognized per group.

### Results

Group 3 RMs Env-binding serum IgG titers remained stable from week 3 to 29 of the priming phase in the absence of a booster immunization (FIG. 2A). After boosting, Group 2 and 3 animals generated similar peak binding antibody titers (2 to 3 weeks post-boost), but Group 3 animals maintained significantly higher Env-binding IgG titers at week 6 post-boost (FIG. 2B).

The quality of the antibody responses was next evaluated in terms of ability to neutralize the tier-2 autologous BG505 pseudovirus. Notably, autologous tier-2 neutralizing antibodies were detectable in all long prime Group 3 animals after only the priming immunization (geometric mean titer [GMT] ˜170 at week 29, FIG. 2C). All animals receiving ED immunization generated robust neutralizing antibody responses post-boost (FIG. 2C-2D); in contrast, only a single animal with conventional bolus immunization adjuvanted with alum had detectable autologous neutralizing antibodies, which were of low titer (˜37, FIG. 7A). Peak observed autologous tier-2 neutralizing antibody GMTs in Group 3 (long prime) were all >2,000 (week 3 post-boost, FIG. 2C). Group 3 autologous tier 2 neutralization titers were 4-fold greater than Group 2 at 6 weeks post-boost (FIG. 2C-2D). The titers described represent the most robust and consistent autologous tier-2 neutralizing antibody responses in RMs after two immunizations in any of the studies (Pauthner, et al., Immunity 46, 1073-1088.e6 (2017), Cirelli, et al., Cell 177, 1153-1171.e28 (2019), Silva, et al., Sci. Immunol. 6, (2021)).

Groups 2 and 3 post-boost sera exhibited some of the broadest observed tier-2 neutralizing antibody specificities elicited by Env trimer immunization, with most animals from Group 3 exhibiting greater breadth than those in Group 2 (FIGS. 2E-2F). The 12-virus neutralization panel was repeated by an independent laboratory, with similar observation of neutralization breadth (FIGS. 7B-7C). In head-to-head neutralization assays, limited tier-2 neutralization breadth was observed with serum from an earlier RM study given ED immunization and two booster immunizations with a similar Env trimer (Olio6) and an earlier ISCOMs adjuvant (SMNP without MPLA) (Cirelli, et al., Cell 177, 1153-1171.e28 (2019)) (FIGS. 7D-7H), and likewise for serum from an RM study with three bolus immunizations of MD39 Env trimer and SMNP (FIG. 7E-7H) (Silva, et al., Sci. Immunol. 6, (2021)).

The vast majority of HIV Env-binding B cells and antibodies are usually directed to immunodominant non-neutralizing epitopes, such as the base of soluble recombinant Env trimers (Cirelli, et al., Cell 177, 1153-1171.e28 (2019), Hu, et al., J. Virol. 89, 10383-10398 (2015), Cottrell, et al., PLoS Pathog. 16, 1-23 (2020), Bianchi, et al., Immunity 49, 288-300.e8 (2018), Havenar-Daughton, et al., Immunol. Rev. 275, 49-61 (2017)). Electron microscopy polyclonal epitope mapping (EMPEM) (Bianchi, et al., Immunity 49, 288-300.e8 (2018)) of circulating antibodies revealed that the number of targeted epitopes correlated with autologous neutralizing antibody titers (FIG. 2G and FIGS. 8A-8C, Table 3). Group 2 and 3 animals generated antibody responses to V5/C3 and V1/V3 epitopes associated with autologous BG505 SHIV protection (Zhao, et al., Cell Rep. 32, 108122 (2020)). Antibody responses in conventional bolus plus alum-immunized animals were largely restricted to the Env trimer base (FIG. 2G). In sum, employing a 12-day ED immunization strategy and vaccine formulation with SMNP was associated with substantially improved epitope breadth and quality of neutralizing antibodies.

TABLE 3
Electron microscopy data bank (EMDB) codes corresponding
to each map generated from EMPEM analysis

| EMDB code | Group | Animal ID | Timepoint | Map | Polyclonal Ab epitopes |
|---|---|---|---|---|---|
| EMD-27609 | 1 | Rh.NJ78 | Wk 13 | Main map | V5/C3 |
| | | | | Additional map 1 | Base |
| EMD-27610 | 1 | Rh.NJ82 | Wk 13 | Main map | Base |
| | | | | Additional map 1 | N611 |
| EMD-27611 | 1 | Rh.NJ95 | Wk 13 | Main map | Base |
| EMD-27612 | 1 | Rh.NK05 | Wk 13 | Main map | Base |
| EMD-27614 | 1 | Rh.NJ79 | Wk 13 | Main map | Base |
| EMD-27615 | 1 | Rh.NJ93 | Wk 13 | Main map | Base |
| | | | | Additional map 1 | V5/C3 |
| EMD-27600 | 2 | Rh.L614 | Wk 13 | Main map | Base, N335/N289, N611 |
| | | | | Additional map 1 | V5/C3 |
| EMD-27601 | 2 | Rh.K409 | Wk 13 | Main map | Base, V5/C3, FP/gp41, N611 |
| EMD-27602 | 2 | Rh.K487 | Wk 13 | Main map | Base, V5/C3, FP/gp41 |
| EMD-27603 | 2 | Rh.DGT5 | Wk 13 | Main map | N335/N289, V1/V3 |
| | | | | Additional map 1 | Base, V5/C3 |
| EMD-27604 | 3 | Rh.K949 | Wk 33 | Main map | Base, N335/N289, N611, V1/V3 |
| | | | | Additional map 1 | V5/C3 |
| EMD-27605 | 3 | Rh.L282 | Wk 33 | Main map | Base, N335/N289, V5/C3, FP/gp41 |
| EMD-27607 | 3 | Rh.DHHT | Wk 33 | Main map | Base, V5/C3, FP/gp41 |
| EMD-27608 | 3 | Rh.DHID | Wk 33 | Main map | Base, N355/N289, V1/V3 |

### Example 3: Six-Month B_{GC} Cells are Highly Functional

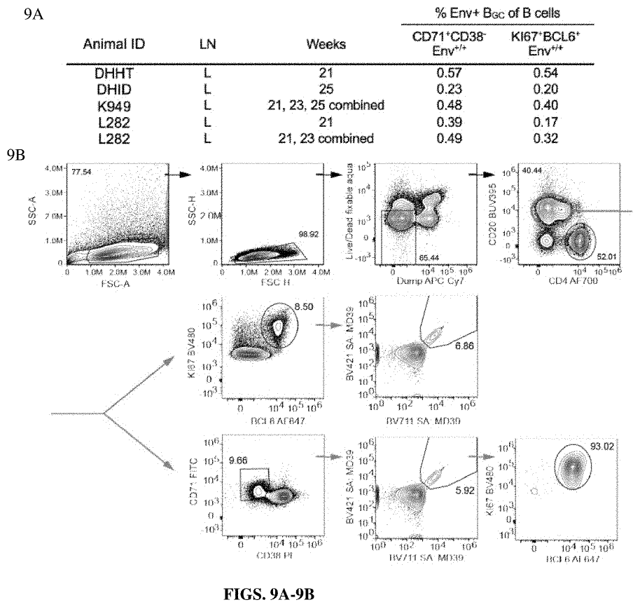

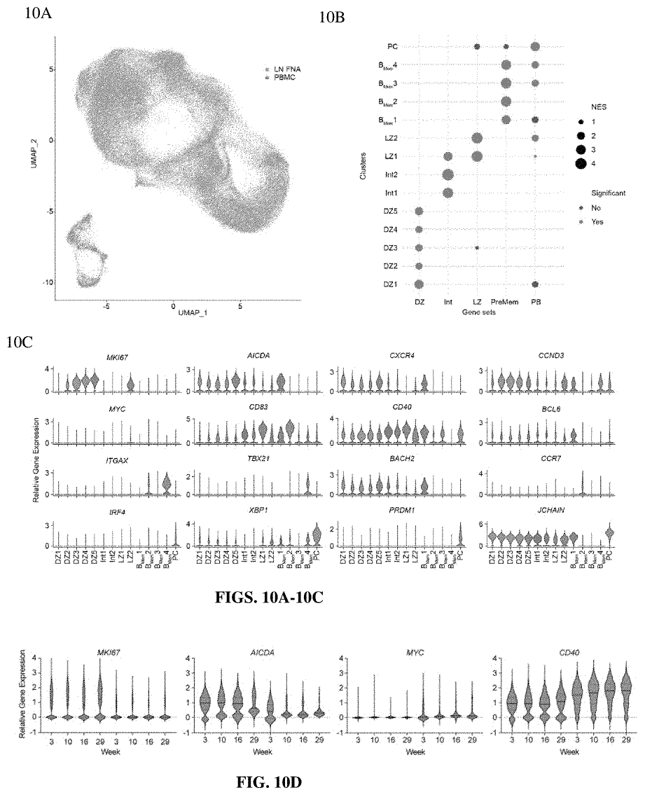

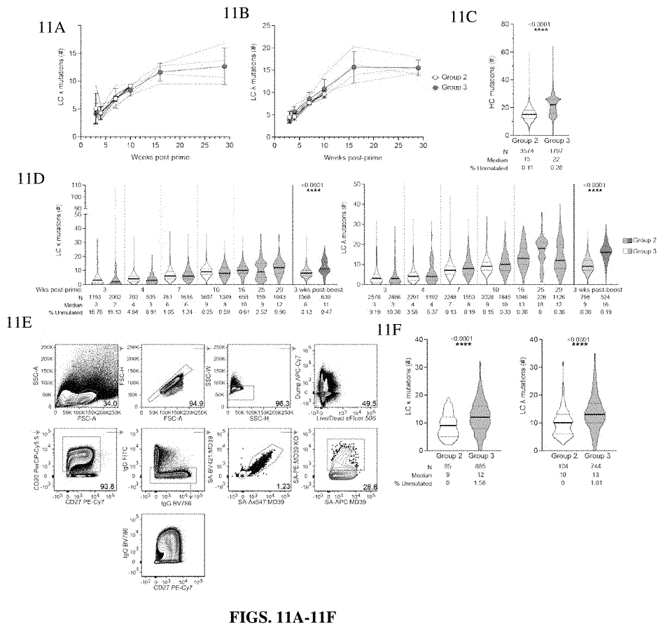

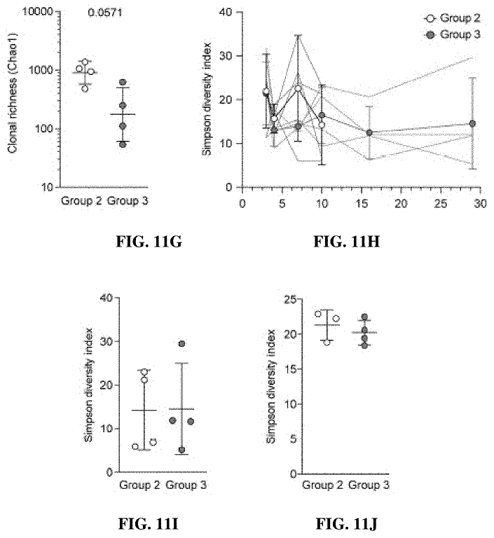

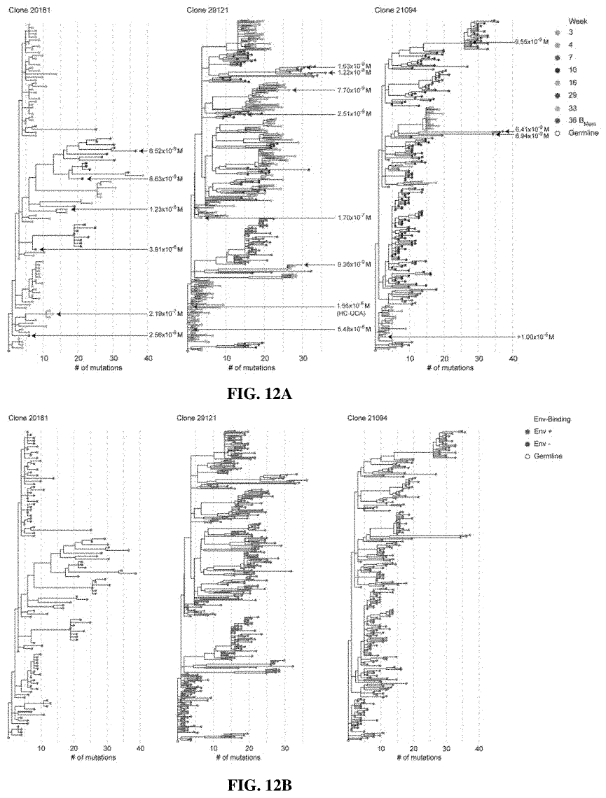

B_{GC} cell characteristics after priming were subsequently interrogated in greater detail to assess their functionality over time, given the apparent presence of continuously active B_{GC} cells for over six months. BCL6 is the lineage-defining transcription factor of B_{GC} cells and is essential for their functionality (Victora & Nussenzweig, Annu. Rev. Immunol. 30, 429-457 (2012)). KI67 (MKI67) marks rapidly dividing cells. LN B cells from month 5 to 6 (week 21-25) were stained for BCL6 and KI67 protein. On average, ˜72% of Env-binding CD71⁺CD38⁻ B_{GC} cells were KI67⁺BCL6⁺ (FIG. 3A-3B and FIG. 9A-9B), indicating retained BGC programming and proliferation for at least six months. To further ascertain the phenotypic and functional characteristics of B_{GC} cells at different time points over the course of six months, single cell transcriptional profiling was done for ˜70,000 cells from LN FNAs of weeks 3, 4, 7, 10, 13, 16, 29, and 33, predominantly consisting of Env-binding B_{GC} cells, as well as peripheral blood sorted Env-binding memory B cells (B_{Mem}) from weeks 16 (Group 2) and 36 (Group 3). Dark zone (DZ) and light zone (LZ) cell clusters were clearly observed among LN B cells when analyzing all time points together (FIGS. 3C-3D, FIGS. 10A-10C). The B_{GC} cell transcriptional profiles were then examined over the course of the Group 3 RMs long priming period (week 3, 7, 16, 29. FIGS. 3D-3H, FIGS. 10A-10C) (Kennedy, et al., Nat. Immunol. doi:10.1038/s41590-020-0660-2 (2020), Holmes, et al., J. Exp. Med. 217, (2020)). LZ and DZ states were sustained across the six-month period (FIG. 3D). Expression of key functional B_{GC} genes MKI67, AICDA, MYC, and CD40 were maintained over time, and were compartmentalized comparably between DZ and LZ cell types at all time points (FIGS. 3E-3H). The ratio of DZ:LZ cells remained relatively consistent over the course of the priming period (FIG. 3I). Overall, antigen-specific B_{GC} cells possessed stable phenotypic characteristics over a six-month period, indicative of long-term maintenance of functional BGC cell properties in the absence of additional immunization.

### Example 4: B_{GC} Cell BCR Evolution for Months in the Absence of Additional Antigen

### Materials and Methods

BCR Sequencing and Processing

A custom RM germline VDJ library was generated using references published by Cirelli et al. (Cirelli, et al., Cell 177, 1153-1171.e28 (2019)), and Bernat et al (Vázquez Bernat, et al., Immunity 54, 355-366.e4 (2021)). CellRanger V3.0 was used to assemble full length V(D)J reads. The constants.py file in the CellRanger VDJ python library was modified to increase the maximum acceptable CDR3 length to 110 NT. CellRanger V6 was used to obtain gene expression counts from sequenced GEX libraries. Libraries were aligned to the Ensemble Mmul10 reference genome, with addition of mitochondrial genes from Mmul9. Sequences were demultiplexed by hashtags using the MULTIseqDemux command in Seurat V4 (Hao, Cell doi:10.1016/j.cell.2021.04.048 (2021)). For HC sequences where both kappa and lambda LC contigs were detected, the B cell was assigned a lambda LC, because lambda LC rearrangement only takes place if the kappa LC is not productive.

Longitudinal Lineage and Somatic Mutation Analysis of BCR Sequences

The VDJ sequence output from CellRanger was further analyzed using packages from the Immcantation portal (Vander Heiden, et al., Bioinformatics 30, 1930-1932 (2014)). An IgBLAST database was built from the custom RM germline VDJ Library. This was then used to parse the 10X V(D)J output from CellRanger into an AIRR community standardized format using the Change-O pipeline to allow for further downstream analysis with the Immcantation portal. Clonal lineages were determined for each animal with DefineClones.py, using the appropriate clustering threshold as determined by the distToNearest command from the SHazaM package in R. Inferred germline V and J sequences from the reference library were added with CreateGermline.py. Because germline D genes sequences and N nucleotide additions cannot be accurately predicted, these were masked from further analysis. The total number of mutations (V and J-genes) for each HC and LC sequence was calculated by counting the number of nucleotide changes between the observed and predicted germline sequences with SHazaM's observe mutation command. For the analysis of total HC mutations, all productive HC contigs were analyzed. For LCs, only contigs paired with HCs were assessed. Sequences where the VH or VL call aligned to alleles IGHV3-100*01, IGHV3-100*01_S4205, IGHV3-100*01_S4375, IGHV3-36*01_S5206, IGHV3-36*01_S6650, IGHV3-NL_11*01_S5714, IGHV4-79-a, IGHV4-NL_1*01_S0419, IGLV1-69, IGLV1-ACR*0 or IGLV2-ABX*01, were found to have an extremely high degree of substituted nucleotides at all timepoints compared to their inferred germline sequences, likely because of poor V gene assignment due to an incomplete V(D)J reference library. These sequences were excluded from further analysis. Only paired HC-LC BCR sequences were analyzed when building clonal trees. Maximum-likelihood lineage trees were built for clonal families with Dowser (Hoehn, et al., Elife 10, (2021)) using the pml method in the GetTrees function. For lineage trees, the length of the branches represents the estimated number of total mutations that have occurred in each HC clone and its most recent common ancestor in lineage, rather than a simple count of nucleotide changes in the germline sequence. Strict criteria for identification of clonal lineages that could be found in both left and right LNs included the following requirements: H-CDR3 length greater than 14, multiple N additions in H-CDR3, greater than 20 cells in total and a matching LC within the lineage. A more lenient set of criteria was also used consisting of the following requirements: H-CDR3 length greater than 10, more than five cells in total and a matching LC within the lineage.

Transcriptomics Analysis

The package Seurat V4 (Hao, Cell doi:10.1016/j.cell.2021.04.048 (2021)) was used for graph-based clustering and visualizations of the gene expression data generated by CellRanger. Initial filtering was conducted on each sample to remove cells expressing <200 or >4500 genes as well as cells with >10% of their transcriptome made up of mitochondrial DNA. Gene expression counts were log normalized via the NormalizeData command A list of common variable genes for across all sample were identified with the SelectIntergrationFeatures function. Expression of these common variable genes was scaled using Principal component analysis (PCA) conducted with RunPCA. Next all samples were integrated together into a single dataset using an RPCA reduction to remove batch effects via the FindlntegrationAnchors and IntergrateData commands Louvian clustering was conducted on the entire integrated dataset with the FindNeighbours and FindCluster functions. Clusters containing large numbers of cells with high levels of mitochondrial DNA as well as clusters with low MS4A1 (CD20) and CD19 expression were excluded from further analysis. Differentially expressed genes were identified in Seurat with the FindMarkers function by running Wilcoxon rank sum test for each cluster against all other clusters. Gene Set Enrichment Analysis (GSEA) was conducted using the fgsea package in R (Korotkevich, et al., Fast gene set enrichment analysis, doi:10.1101/060012 (2021), Sergushichev, bioRxiv 060012 (2016)). Differentially expressed genes from previously identified human LZ, DZ, Intermediate, PreMem and Plasmablast subsets were taken from Holmes et al. (Holmes, et al., J. Exp. Med. 217, (2020)) and combined to create the gene sets used for GSEA. LZ1 and DZ5 were selected as representative light-zone and dark-zone clusters, respectively, based on GSEA scores.

### Results

To directly assess functionality of the GCs over these extended time periods, multiple experimental approaches were employed comparing Groups 2 and 3 RMs. BCR sequencing of Env-binding LN FNA-derived BGC cells was performed from nine different timepoints to assess SHM over time, as well as clonal diversity and mutational patterns in clonal lineages (FIGS. 4A-4L and FIGS. 11A-14C). Env-binding B_{GC} cell heavy chain (HC) nucleotide (NT) mutations increased significantly between week 3 and 10 (Mann-Whitney, P<0.0001 for both Group 2 & 3, FIG. 4A-4B). Notably, B_{GC} cells continued to accumulate mutations in the absence of another immunization through week 29 in Group 3 RMs, at which point the median number of HC mutations was 17, with the top 25% of B_{GC} cells containing 22 to 45 HC mutations (FIG. 4A-4B). The difference in SHM in the long prime (week 29) versus 10-week prime was highly significant (Mann-Whitney, P<0.0001, FIG. 4B; P<0.0009, FIG. 4C), and the difference in median mutations between weeks 10 and 29 was nearly as great as the difference between weeks 3 and 10, indicative of robust GC functionality continuing through at least week 29 (FIGS. 4A-4C). Env-binding BGC cells showed a gradual reduction in the diversity of clones (population diversity) over time (FIGS. 4D-4E), further indicative of ongoing competitive pressure. The proportion of unmutated Env-binding B_{GC} cells dropped over time, with 0.19-0.42% unmutated cells by week 7 (FIG. 4B). Substantial mutations were also observed in the light chain (LC) sequences over time, with comparable patterns to the HCs (FIGS. 11A-11B).

After the booster immunization, HC mutations increased in Env-binding B_{GC} cells of both Group 2 and 3 RMs, with the highest overall number of mutations in the long primed RMs (week 3 post-boost HC median mutations=13 vs. 20, FIGS. 4B-4C, FIG. 11C). Comparable observations were made for LC mutations (FIG. 11D).

Pre-boost Env-binding B_{Mem} cell (CD20⁺IgD⁻Env^{+/+}) frequencies in peripheral blood were equivalent in RMs from Groups 2 and 3 (FIG. 4F, FIG. 11E). Boosting increased Env-binding B_{Mem} frequencies in both groups (FIG. 4F). RMs with the long prime had more highly mutated B_{Mem} cells and greater clonal richness among B_{Mem} cells (FIGS. 4G-4H, FIG. 11F). This was also reflected in a significant shift away from immunodominant base-binding Env-specific B_{Mem} cells of the long primed group compared to Group 2 (FIG. 4I), a phenomenon that was also reflected in the circulating antibody titers (FIG. 4J).

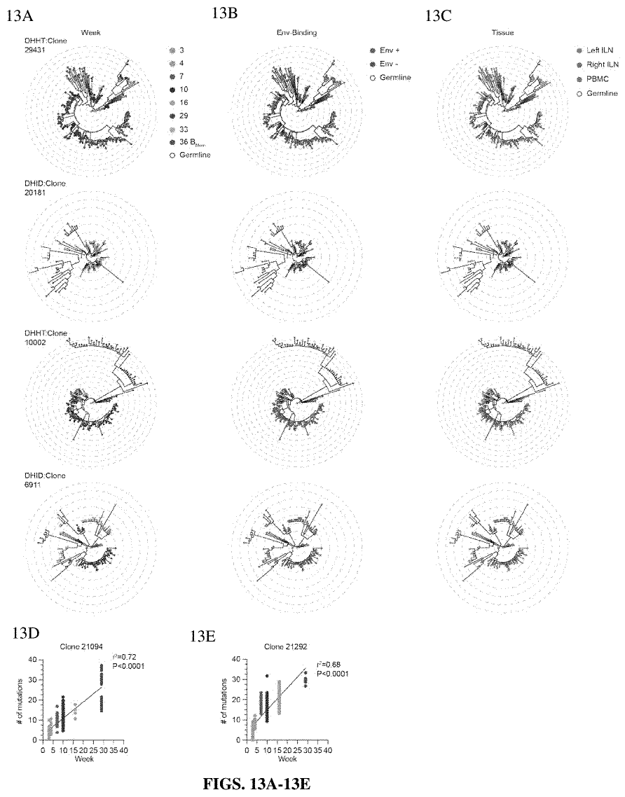

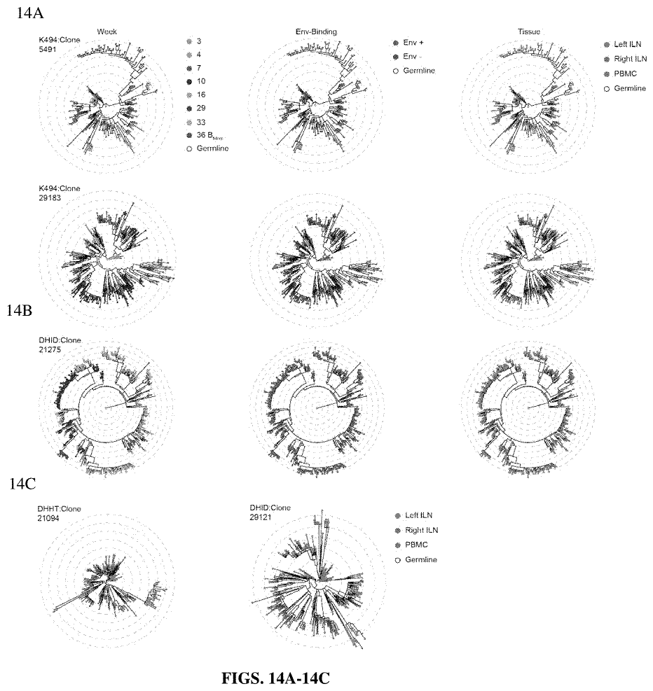

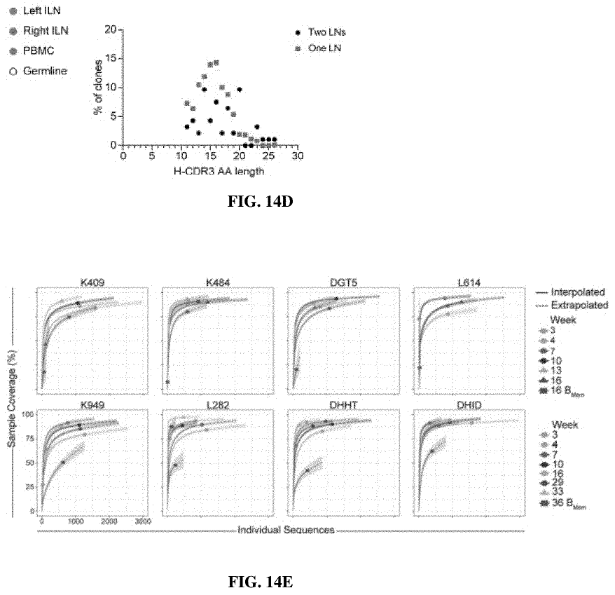

Clonal lineage analysis of paired BCR sequences was used to examine GC duration and functionality over months after a single immunization. Many clonal lineages were identified with B_{GC} clones observed at the first and last LN FNA timepoint post-prime, with extensive SHMs in-between (FIG. 4K, FIGS. 12A-12B, 14C). Some clonal lineages were identified with B_{GC} clones observed at every, or nearly every, LN FNA timepoint, providing direct evidence of B_{GC} cell persistence for 29 weeks (FIG. 4K, FIGS. 12A-12B, 13A, 14A-14B). Diversification of clones in clonal lineages was apparent. Daughter clones most evolutionarily divergent from the germline were typically present at late time points (mutations to time correlation. R²=0.72, P<0.001, lineage 21094; R²=0.68, P<0.001, lineage 29121. FIG. 4k, FIGS. 12A-12B, 13D-13E). For some lineages such as 5491 and 29183, the majority of early B_{GC} cell clones did not bind Env by flow cytometry (Env^{-/-}), but the majority of B_{GC} clones from week 16+, and B_{Mem} clones from week 36, bound Env by flow cytometry (Env^{-/-}), indicative of a clone that started with low affinity to Env and affinity matured (FIG. 14A).

To directly assess affinity maturation within these lineages, monoclonal antibodies from several time points in three large clonal lineages were generated. An early monoclonal antibody from lineage 20181 had relatively low affinity to MD39 (week 4, dissociation constant (K_{d})=2.19×10⁻⁷ M). An increase in affinity of roughly 17-fold (K_{d}=1.23×10⁻⁸ M) was observed by week 16 and a further (roughly) twofold increase by week 29 (K_{d}=6.52×10⁻⁹ M), representing around a 30-fold overall increase in affinity (FIGS. 12A-12B). In lineage 29121, the antibody with the lowest affinity at week 3 (K_{d}=1.55×10⁻⁶ M) used an HC identical to the amino acid sequence of the unmutated common ancestor (UCA) (FIGS. 12A-12B). In this lineage, almost one-third of the cells found at week 3 did not bind MD39 via flow cytometry, including the UCA HC clone (FIGS. 12A-12B), which indicates that this lineage started with a very weak affinity and was still able to participate substantially in the GC reaction. In the same lineage, antibodies were isolated at weeks 16 and 29 with affinities about 200-fold (K_{d}=7.70×10⁻⁹ M) and 950-fold (K_{d}=1.63×10⁻⁹ M) higher than the week 3 HC UCA clone (FIGS. 12A-12B). Finally, a low-affinity lineage 21094 antibody (week 3, K_{d}>1.00×10⁻⁵ M; Env−/− by flow cytometry) had gained over a 1,000-fold improvement in affinity by week 29 (K_{d}=9.55×10⁻⁹ M, 6.49×10⁻⁹ M) (FIGS. 12A-12B). Altogether, these data strongly support the conclusion that substantial affinity maturation occurs over the course of the 6-month priming period, leading in some cases to B_{GC} cells with around 1,000-fold improved affinity.

B_{Mem} cells were commonly represented in the clonal lineage trees among multiple sub-lineages, including amongst the most mutated branches observed at late LN FNA B_{GC} time points (FIG. 4K; FIGS. 12A-14C), demonstrating that the GCs productively output B_{Mem} cells and seeded the peripheral B_{Mem} cell compartment throughout 29 weeks. Clonal lineages with other interesting features were observed from the long GCs, including lineage 21275 which was almost exclusively IgM from week 3 to 29 (FIG. 14B).

There were clonal lineages with clones detected in both the left ILN and right ILN at different time points during the long GCs (FIGS. 13C, 14A-14C). Out of 169 clonal lineages passing stringent criteria (HC complementarity determining region 3 [H-CDR3] length ≥14, multiple N-additions in the H-CDR3, ≥20 cells total), 11 lineages were observed in both LNs (≥10 cells per LN) at different time points, providing evidence that B_{Mem} cells generated from GCs in one LN can exit, recirculate, and enter ongoing GCs in another LN. Using less restrictive criteria of clonal lineages containing at least five cells with H-CDR3 length greater than 10 and a matching LC, it was observed that 54 out of 889 clonal lineages could be found in bilateral LNs. In sum, key features observed in numerous antigen-specific clonal lineages provide direct evidence of B_{GC} cell persistence for 29 weeks, continuous accumulation of somatic mutations at substantial rates, affinity maturation, and seeding the peripheral B_{Mem} cell compartment, which together show that under select conditions, GCs are able to undergo clonal competition evolution for extremely long durations without new antigen exposure.

### SUMMARY

A 12- to 14-day slow-delivery immunization regimen can result in substantially greater capture of vaccine antigens by stromal follicular dendritic cells (Cirelli, K. M. et al., Cell 177, 1153-1171 (2019)). Observation of GCs for over 6 months indicates that endocytic recycling of immune complexes by follicular dendritic cells (Heesters, et al., Immunity 38, 1164-1175 (2013)) can be efficient at maintaining proteins in GCs and protecting them from damage. One possible mechanism of slow-delivery enhancement of GCs is improved immune complex formation, due to antigen supply after the start of the antibody response. Given the immunodominance of antibody responses to the non-neutralizing base of the Env trimer after conventional immunizations, and epitope diversification to non-base epitopes in slow-delivery immunizations shown herein, it is believed that immune complexes with Env under slow-delivery conditions are primarily composed of base-binding antibodies, which shield the base of the Env trimer in GCs and orient the trimers to better demonstrate neutralizing epitopes antipodal to the base, thereby enriching for neutralizing antibody B cells. This is further illustrated by the shift away from base-directed immunodominance in Bₘₑₘ cells during the long prime. Thus, the improved autologous and heterologous neutralization noted in group 3 monkeys is probably partly owing to the diversity of B cells recruited and partly due to increased affinity maturation from extensive GC responses. These outcomes are consistent with a conclusion that immunodominance is a major factor that effects neutralizing antibody responses to candidate HIV vaccines, and the immunodominance of non-neutralizing antibody responses can be overcome by recruiting more diverse B cells to GCs and providing those cells with sufficient time for affinity maturation to become competitive as Bₘₑₘ cells with non-neutralizing Bₘₑₘ cells at the time of booster immunization (Havenar-Daughton, et al., Immunol. Rev. 275, 49-61 (2017).).

The appearance of neutralizing titres in the long-prime group 3 between weeks 20 and 29 after prime is indicative of continued affinity maturation, leading to GC output of new plasma cells that can produce neutralizing antibodies months after the initial immunization. Regarding the kinetics of B cell memory development, the data are inconsistent with a model of predominantly early Bₘₑₘ cell production from continuing GCs. If that were the case, median Bₘₑₘ cell HC SHMs would be low (for example, two to five mutations), corresponding to early B_{GC} cells (3-4 weeks). By contrast, median Bₘₑₘ cell HC SHM was 17 at week 36 and less than 15% of Bₘₑₘ cells had fewer than 10 mutations. The simplest interpretation of these observations is that early Bmem cells may re-enter GCs to undergo further SHM and affinity maturation. The finding of shared B_{GC} clones between left and right LNs and circulating Bₘₑₘ cells is most consistent with this Bₘₑₘ GC re-entry model. In addition, these data are inconsistent with a model of B_{GC} cell extinction after around 1 month and dominant replacement by incoming naive B cells, because unmutated B cells were rare in GCs after 1 month and median B_{GC} cell SHM levels continued to increase over 6 months. B_{GC} cell clonal lineages were also observed to span the full 6 months after priming Overall, the data are consistent with ongoing production or recycling of Bₘₑₘ cells and the production of plasma cells from ongoing GCs over the course of the priming period. The results show that GCs can persist for more than 6 months in response to a priming immunization, with several notable outcomes. These findings indicate that patience can have value in allowing antibody diversification and evolution in GCs over extended periods of time, and this long-prime, slow-delivery immunization approach holds promise for difficult vaccine targets.

Lee, et al., “Long-primed germinal centres with enduring affinity maturation and clonal migration”, Nature, 609:998-1020 (2022), https://doi.org/10.1038/s41586-022-05216-9 is specifically incorporated by reference herein in its entirety.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of inducing an immune response in a subject in need thereof comprising administering the subject an effective amount of an antigen and adjuvant to induce an immune response against an antigen, the method comprising two or more of (i) slow prime delivery of antigen and/or adjuvant, a (ii) temporally delayed 2nd immunization, and (iii) a robust adjuvant.

2. The method of claim 1, comprising all three of (i), (ii), and (iii).

3. The method of claim 1, comprising (i) wherein (i) comprises temporally extended exposure of antigen, adjuvant, or the combination thereof.

4. The method of claim 3, comprising (i) wherein (i) comprises temporally extended exposure to both antigen and adjuvant.

5. The method of claim 3, comprising (i) wherein (i) comprises prime delivery of the antigen and/or adjuvant using one or more of repeated administrations, infusion optionally by osmotic pump (OP), escalating dosing (ED), and sustained release carriers to increase the duration of antigen and/or adjuvant in the subject.

6. The method of claim 5, wherein the infusion comprises continuous delivery of antigen and/or adjuvant for hours, days or weeks.

7. The method of claim 6, wherein the infusion is for 5-21 days inclusive, or any specific number therebetween.

8. The method of claim 6, wherein the continuous delivery is of a consistent discrete dose or increasing dose of antigen and/or adjuvant.

9. The method of claim 5, wherein escalating dosing comprises administering the subject two or more doses at temporally increasing doses of antigen and/or adjuvant.

10. The method of claim 9, wherein the increase in dosing is by discrete or continuous administration.

11. The method of claim 10 comprising 2-21 discrete administrations.

12. The method of claim 9, wherein different doses are administered hours or days apart.

13. The method of claim 12, wherein 5-10 doses, optionally 7 doses are administered about every other day.

14. The method of claim 9, wherein each subsequent dose is higher than the preceding dose.

15. The method claim 5, wherein the antigen and/or adjuvant are in a sustained release formulation.

16. The method of claim 1, comprising (i) wherein (i) comprises administering the antigen and adjuvant in the same or different admixtures.

17. The method of claim 16, where the antigen and adjuvant are in different admixtures administered by the same or different schedules.

18. The method of claim 17, wherein the antigen and adjuvant are administered by different routes of administration.

19. The method of claim 1, comprising (i) wherein (i) comprises administering the antigen and/or adjuvant by subcutaneous, intramuscular, or intravenous injection or infusion.

20. The method of claim 1, comprising (i) wherein (i) comprises administering the antigen and adjuvant in the same admixture or different admixtures by subcutaneous administration according to the same schedule.

21. The method of claim 1, comprising (ii) wherein (ii) comprises administering one or more boost doses is between 11 and 35 weeks, or between 15 and 40 weeks, or between 20 and 35 weeks after the start or the conclusion of the prime administration of antigen and/or adjuvant.

22. The method of claim 1, comprising (ii) wherein (ii) comprises administering one or more boost doses 25, 26, 27, 28, 29, 30, 32, 32, 33, 34, or 35 weeks after the start of prime dosing.

23. The method of claim 1, comprising (ii) wherein (ii) comprises administering the antigen and/or adjuvant by a single bolus dose or temporally extended exposure of antigen, adjuvant, or the combination thereof.

24. The method of claim 1, comprising (ii) wherein (ii) comprises administering the antigen and adjuvant in the same or different admixtures.

25. The method of claim 1, where the antigen and adjuvant are in different admixtures administered by the same or different schedules.

26. The method of claim 25, wherein the antigen and adjuvant are administered by different routes of administration.

27. The method of claim 1, comprising (ii) wherein (ii) comprises administering the antigen and/or adjuvant by subcutaneous, intramuscular, or intravenous injection or infusion.

28. The method of claim 1, comprising (ii) wherein (ii) comprises administering the antigen and adjuvant in the same admixture or different admixtures by subcutaneous administration according the same schedule.

29. The method of claim 1, wherein the antigen and/or adjuvant are administered in an effective amount and/or manner that increases GC response levels and/or duration optionally relative to traditional prime and short boost of antigen and an adjuvant consisting of alum.

30. The method of claim 1, wherein the antigen and/or adjuvant are administered in effective amount and/or manner that reduces immunodominance and/or increases neutralizing antibodies optionally relative to traditional prime and short boost of antigen and an adjuvant consisting of alum.

31. The method of claim 1, comprising (iii) wherein (iii) comprises a non-liposome, non-micelle particle comprising of a lipid, a sterol, a saponin, and an additional adjuvant.

32. The method of claim 31, wherein the particle is a porous, cage-like nanoparticle.

33. The method of claim 32, wherein the porous, cage-like nanoparticle is about 30 nm to about 60 nm.

34. The method of claim 33 comprising lipid:additional adjuvant:sterol:saponin molar ratio of 2.5:1:10:10, or a variation thereof wherein the molar ratio of lipid, additional adjuvant, sterol, saponin or any combination thereof is increased or decreased by any value between about 0 and about 3.

35. The method of claim 34, wherein the lipid is a phospholipid, the additional adjuvant is a TLR4 agonist, the sterol is cholesterol or a derivative thereof, and the saponin is Quil A or submixture or pure saponin separated therefrom.

36. The method of claim 35, wherein the phospholipid is 2-Dipalmitoyl-sn-glycero-3-phosphocholine (DPPC), the TLR4 agonist is a natural or synthetic monophosphoryl lipid A (MPLA), the sterol is cholesterol, and the saponin is Quil A or Q-21.

37. The method of claim 1, wherein the antigen is derived from a virus, bacterium, parasite, plant, protozoan, fungus, tissue or transformed cell such as a cancer.

38. The method of claim 37, wherein the virus is a coronavirus.

39. The method of claim 38, wherein the coronavirus is a severe acute respiratory syndrome coronavirus 2 (SARS-CoV-2).

40. The method of claim 39, wherein the antigen comprises the SARS-CoV-2 spike (S) protein or a fragment or epitope(s) thereof.
